(19) United States
(12) Patent Application Publication
(10) Pub. No.: US 2005/0032075 A1
(43) Pub. Date: Feb. 10, 2005

(54) Method of detecting primer extension reaction, method of discriminating base type, device for discriminating base type, device for detecting pyrophosphate, method of detecting nucleic acid and tip for introducing sample solution

(76) Inventors: Hidenobu Yaku, Osaka (JP); Tetsuo Yukimasa, Nara-shi (JP); Hiroaki Oka, Osaka (JP)

Correspondence Address: MCDERMOTT WILL & EMERY LLP, 600 13TH STREET, N.W., WASHINGTON, DC, 20005-3096, US

(21) Appl. No.: 10/674,787

(22) Filed: Oct. 1, 2003

(30) Foreign Application Priority Data
Oct. 1, 2002 (JP) 2002-288837

(51) Int. Cl. C12Q 1/68

(52) U.S. Cl. 435/6

(57) ABSTRACT

Convenient techniques for discriminating the base type in a base sequence of a nucleic acid are provided. The technique includes the step (a) of preparing a sample solution containing a nucleic acid, a primer having a base sequence that includes a complementary binding region which complementarily binds to the nucleic acid, and a nucleotide; the step (b) of allowing the sample solution to stand under a condition to cause an extension reaction of the primer, and producing pyrophosphate when the extension reaction is caused; the step (c) of bringing the sample solution into contact with the front face of a H⁺ hardly permeable membrane having H⁺-pyrophosphatase, which penetrates from front to back of the membrane, of which active site that hydrolyzes pyrophosphate being exposed to the front face; the step (d) of measuring the H⁺ concentration of at least either one of the solution at the front face side of the H⁺ hardly permeable membrane or the solution at the back face side of the H⁺ hardly permeable membrane, in a state where the H⁺-pyrophosphatase is immersed in the solution; the step (e) of detecting the extension reaction on the basis of the result of measurement in the step (d) ; and the step (f) of discriminating the base type in the base sequence of the nucleic acid on the basis of the result of detection in the step (e).

### BACKGROUND OF THE INVENTION

1. Field of the Invention

The present invention relates to a method of detecting an extension reaction in which an extension reaction of a primer is detected, a method of discriminating a base type in which the base type in a base sequence of a nucleic acid is discriminated, a device for discriminating a base type in which the base type in a base sequence of a nucleic acid is discriminated, a device for detecting pyrophosphate, a method of detecting a nucleic acid and a tip for introducing a sample solution.

2. Description of the Related Art

(First Conventional Technology)

Techniques for determining the presence/absence of a nucleic acid having a particular base sequence are very important techniques. For example, they are essential in diagnoses of hereditary diseases; inspections of food contamination with bacteria, viruses and the like; and inspections of infection of a human body with bacteria, viruses and the like.

Hereditary diseases such as severe combined immunodeficiency disease, familial hypercholesterolemia and the like are elucidated to result from deficiency of a particular gene. Hence, the presence/absence of a hereditary disease can be diagnosed by examining the presence/absence of a gene having a particular base sequence which may cause the hereditary disease as described above.

In recent years, there arises a social problem of food contamination by Escherichia coli O157 and the like. For inspections of food contamination with such bacteria, viruses and the like, the presence/absence of the contamination can be determined by analyzing the presence/absence of a base sequence of a DNA or RNA that is inherent to a bacterium or virus suspected of the contamination. The same applies to inspections of infection of a human body.

In general, the detection techniques of a base sequence of a particular nucleic acid as described above requires very high sensitivity of detection because the nucleic acid, as a sample, which contains the particular base sequence is included in a slight amount in many cases. At present, the detection techniques that are most commonly used are techniques in which an amplification method of a nucleic acid having a target base sequence is utilized. For example, they include a PCR method, ICAN method, LCR method, SDA method, LAMP method and the like. By way of these amplification methods of a nucleic acid, a nucleic acid having a target base sequence in a sample is amplified in a large quantity, and the nucleic acid having the target nucleic acid is detected. The aforementioned amplification method can readily amplify a nucleic acid having a target base sequence. However, there exist some defects in the method of detecting an amplified nucleic acid having a target base sequence.

One of the most versatile methods of detecting an amplified nucleic acid having a target base sequence is a method in which the amplified nucleic acid having the target base sequence is separated by electrophoresis, and thereafter a fluorescent intercalating agent such as ethidium bromide or the like is used. Although this method is convenient, on the other hand, special attention must be paid during handling because the fluorescent intercalating agent is a carcinogen.

Further, other exemplary method may be a dot blot method. In the dot blot method, an amplified double stranded DNA or RNA having a target base sequence is denaturated into a single stranded DNA or RNA by a heat treatment, and fixed onto a membrane such as nylon. Subsequently, it is subjected to radiolebelling or fluorescent lebelling, followed by hybridization to a nucleic acid probe that specifically reacts with the single stranded DNA or RNA described above on the membrane. Finally, detection of the amplified DNA or RNA having the target base sequence is executed by carrying out the detection of the radiolebelling or fluorescent lebelling. However, in this method, time period of 1 to five days is usually consumed when a radio labelled nucleic acid probe is employed. Further, also in instances where a fluorescent labelled nucleic acid probe is employed, several hours to ten and several hours are also required. In addition, a labelled nucleic acid probe must be prepared for each amplified nucleic acid having the target base sequence, and a burden has been thereby imposed.

The PCR method is a technique in which a nucleic acid having a target base sequence is amplified while repeating a DNA extension reaction from a primer, with the use of DNA polymerase, in general (hereinafter, referred to as primer extension reaction). Application of use of the primer extension reaction is not limited to the detection of a nucleic acid having a target base sequence.

Recently, it has been elucidated that polymorphism of just one base pair in a base sequence, what is called SNP (Single Nucleotide Polymorphism: polymorphism of a single base), affects susceptibility to a disease such as diabetes, hypertension or the like, efficacy of a drug, or the like. Therefore, great importance has been put on a SNP typing technique in which a SNP pattern of each individual is analyzed. Moreover, cases in which a substitution of only one base pair in a base sequence within a genomic DNA becomes the cause of a serious disease have been known. Accordingly, analyses of the presence/absence of such a substitution of a single base pair have also become of great importance. The SNP typing technique is also effective in the discrimination of the presence/absence of such a substitution of a single base pair.

Currently, a variety of SNP typing techniques have been developed or already put into practice. In one of the most convenient techniques among those techniques, a primer extension reaction has been utilized. In this technique, SNP typing is executed by determining whether or not a primer extension reaction is caused.

For the present, discrimination techniques of the base type of a SNP site in which a primer extension reaction is utilized can be generally classified into two groups. One includes primer extension reaction-utilizing methods where 4 kinds of dNTPs (dATP, dCTP, dGTP, dTTP) are used. Another includes primer extension reaction-utilizing methods where one kind of dNTP or ddNTP alone is used.

The primer extension reaction-utilizing method where 4 kinds of dNTPs are used is explained with reference to FIG. 19 and FIG. 20. In this method, a primer that has a complementary base sequence to the base sequence that is adjacent to the SNP site of a target DNA, and that results in a difference of progress of the extension reactions depending on the base type of the SNP site of the target DNA (hereinafter, referred to as typing primer) is used. Specifically, the steps explained below are carried out.

First, in the step illustrated in FIG. 19 (a), a sample solution containing a target DNA 1 having a SNP site S1 is prepared. Similarly, in the step illustrated in FIG. 20 (a), a sample solution containing a target DNA 2 having a SNP site S2 is prepared.

Next, in the step illustrated in FIG. 19 (b), the DNA 1 is converted to single stranded DNAs 3 and 4 by thermal denaturation or the like. Similarly, in the step illustrated in FIG. 20 (b), the DNA 2 is converted to single stranded DNAs 5 and 6 by thermal denaturation or the like.

Next, in the step illustrated in FIG. 19 (c), to the sample solution containing the single stranded DNAs 3 and 4, are added a typing primer 7, DNA polymerase 8 and 4 kinds of dNTPs. Similarly, in the step illustrated in FIG. 20 (c), to the sample solution containing the single stranded DNAs 5 and 6, are added a typing primer 7, DNA polymerase 8 and 4 kinds of dNTPs. The typing primer 7 herein is designed such that it completely hybridizes to the 3′ end sided region from the SNP site of the single stranded DNAs 4 and 6, except for the base at its 3′ end (in this case, thymine (hereinafter, denoted as T)).

In the step illustrated in FIG. 19 (c), the typing primer 7 completely hybridizes to the single stranded DNA 4 having the SNP site S1 of adenine (hereinafter, denoted as A). Thus, in the step illustrated in FIG. 19 (d), a primer extension reaction is caused, thereby consuming dNTP by the DNA polymerase 8.

On the other hand, in the step illustrated in FIG. 20 (c), only the base at the 3′ end of the typing primer 7 (T) can not hybridize to the single stranded DNA 6 having the SNP site S2 of guanine (hereinafter, denoted as G). Hence, in the step illustrated in FIG. 20 (d), a normal primer extension reaction hardly occurs. Therefore, dNTP is hardly consumed.

Accordingly, discrimination of a base at the SNP site is permitted by analyzing the difference of progress of these extension reactions. In such a manner, discrimination of the base at a SNP site is executed on the basis that a primer extension reaction occurs or not. In this method, similar analysis can be effected also in cases where the base at the SNP site can be of 3 types or 4 types, when typing primers corresponding respectively thereto are provided.

In regard to the typing primer, ones other than the primer having the 3′ end base to match to the base of the SNP site of the DNA, as described above, have been also developed. For example, ASP (Allele Specific Primer) developed by Toyobo Co., Ltd. is included (see, web site of Toyobo Co., Ltd., retrieved on Oct. 1, 2002, URL (http://www.toyobo.co.jp/seihin/xr/product/custom/snps/s nps.html)). ASP is a primer designed such that it has the second base from its 3′ end corresponding to the SNP site, and in addition, the third base from its 3′ end being certainly non complementary to the target base.

It is reported that by using ASP together with a type DNA polymerase having potent calibrating activity, more accurate discrimination of the base type of a SNP site than the methods illustrated in FIG. 19 and FIG. 20 as described above is enabled. More specifically, when the SNP site is complementary to the second base from the 3′ end of ASP, a favorable extension reaction is caused, but when it is not complementary thereto, the extension reaction is not properly caused. Furthermore, the difference of progress of the extension reactions between the cases with and without the occurrence of the extension reaction has been reported to be greater than the methods illustrated in FIG. 19 and FIG. 20 as described above.

Next, the primer extension reaction-utilizing method where 1 kind of dNTP (or ddNTP) is used is explained with reference to FIG. 21 and FIG. 22. In this method, the extension reaction is performed using a primer designed such that it hybridizes to a region that is adjacent to the SNP site in a target single stranded DNA. In other words, a site that corresponds to the SNP site is not present in the primer sequence. Specifically, the steps explained below are carried out.

First, in the step illustrated in FIG. 21 (a), a sample solution containing a target DNA 1 having a SNP site S1 is prepared. Similarly, in the step illustrated in FIG. 22 (a), a sample solution containing a target DNA 2 having a SNP site S2 is prepared.

Next, in the step illustrated in FIG. 21 (b), the DNA 1 is converted to single stranded DNAs 3 and 4 by thermal denaturation or the like. Similarly, in the step illustrated in FIG. 22 (b), the DNA 2 is converted to single stranded DNAs 5 and 6 by thermal denaturation or the like.

Next, in the step illustrated in FIG. 21 (c), to the sample solution containing the single stranded DNAs 3 and 4, are added a primer 9, DNA polymerase 8 and dCTP (or ddCTP). Similarly, in the step illustrated in FIG. 22 (c), to the sample solution containing the single stranded DNAs 5 and 6, are added a primer 9, DNA polymerase 8 and dCTP (or ddCTP) The primer 9 herein is designed such that it completely hybridizes to a region that is adjacent to the 3′ end side from the SNP site of the single stranded DNAs 4 and 6. Therefore, the primer 9 completely hybridizes to the single stranded DNAs 4, 6.

Next, in the step illustrated in FIG. 21 (d), a primer extension reaction is not caused because the SNP site S1 of the single stranded DNA 4 is A, and only dCTP (or ddCTP) is supplied. Thus, dCTP (or ddCTP) is hardly consumed by the DNA polymerase 8.

On the other hand, in the step illustrated in FIG. 22 (d), a normal primer extension reaction is caused through the supply of dCTP (or ddCTP) because the SNP site S2 in the single stranded DNA 6 is G. Accordingly, dCTP (or ddCTP) is consumed by the DNA polymerase 8.

In cases where the base at the SNP site can be of 3 types or 4 types, similar analyses are allowed by using dNTP or ddNTP that corresponds thereto, respectively.

Thus, in the method in which one kind dNTP or ddNTP alone, differently from the method in which all 4 kinds of dNTPs described above are used, only approximately one to several bases are generally added to the primer when dNTP is used, whilst only one base is added to the primer when ddNTP is used. Therefore, it is quite difficult to detect the difference of progress of the extension reactions. Then, in the section of Summary of WO98/28440, and the section of Summary of WO98/13523, a method is employed in which pyrophosphate that is produced during the progress of the primer extension reaction is converted to ATP, and the amount of pyrophosphate is thereafter measured utilizing a luciferase reaction, for the purpose of detecting the difference of progress of the extension reactions. Advantages of the method in which one kind of dNTP alone is used include the aspect that discrimination is enabled of not only the SNP site but also the base sequence in the vicinity of the SNP site, by repeating the steps according to each step illustrated in FIG. 21 or FIG. 22, depending on how the primer is designed.

As described hereinabove, there have existed several kinds of discrimination techniques of the base type of a SNP site in which a primer extension reaction is utilized, however, in any one of the discrimination techniques of the base type of a SNP site, the aspect that discrimination of the base type of a SNP site is effected by analyzing the difference of progress of the primer extension reactions is common.

Such discrimination techniques of the base type of a SNP site are extremely useful techniques which can be applied to the discrimination of not alone so called SNP site, but of a desired particular base. It is highly possible that they are utilized routinely at a variety of hospitals irrespective of either large or small scale, in the near future. Therefore, methods which allow for the analysis of a difference of primer extension reactions in safer and more accurate manner have been needed.

(Second Conventional Technology)

Pyrophosphate has been known to greatly participate in enzyme reactions in cells. For example, in a process of synthesis of a protein, pyrophosphate is produced during a reaction in which an amino acid forms an aminoacyl tRNA via aminoacyl adenylate. Further, in a process of synthesis of starch found in e.g., plants and the like, pyrophosphate is produced when ADP-glucose is produced by the reaction between glucose-1-phosphate and ATP. Apart from them, it has been known that pyrophosphate participates in various enzyme reactions. Therefore, techniques for quantitatively determining pyrophosphate are important techniques upon analyses of cellular states, enzyme reactions as described above, or the like.

In JP-A No. 61-12300, three kinds of methods of detecting pyrophosphate in which an enzyme is utilized are disclosed. Those are explained below.

In the first method, pyrophosphate is brought into the action of pyruvate orthophosphate dikinase in the presence of phosphoenol pyruvate and adenosine monophosphate. Because pyruvic acid is produced by this reaction, the amount of pyrophosphate can be derived by calculation through measuring the amount of the pyruvic acid. As methods of measuring the amount of pyruvic acid, two kinds of methods have been proposed. One is a method in which decrease of NADH is colorimetrically determined upon reduction of pyruvic acid with NADH utilizing a catalytic action of lactate dehydrogenase. In another method, hydrogen peroxide, which is produced by bringing thus produced pyruvic acid into the action of pyruvate oxidase, is introduced to a pigment to allow for a calorimetric determination.

In the second method, pyrophosphate is brought into the action of glycerol-3-phosphate cytidyl transferase in the presence of cytidine diphosphorus glycerol. Glycerol triphosphate is produced by this reaction. Therefore, the amount of pyrophosphate can be derived by calculation through measuring the amount of thus produced glycerol triphosphate. As methods of measuring the amount of glycerol triphosphate, two kinds of methods have been proposed. One is a method in which increase of NAD(P)H is calorimetrically determined uponoxidation of glycerol triphosphatewithNAD(P) utilizing a catalytic action of glycerol-3-phosphate dehydrogenase. In another method, hydrogen peroxide, which is produced by bringing thus producedb glycerol triphosphate into the action of glycerol-3-phosphate oxidase, is introduced to a pigment to allow for a calorimetric determination. Another is a method in which calorimetric determination is carried out through introducing hydrogen peroxide, which is produced by bringing thus produced, to a pigment.

In the third method, pyrophosphate is brought into the action of ribitol-5-phosphate cytidyl transferase in the presence of cytidine diphosphate ribitol. Because D-ribitol-5-phosphate is produced by this reaction, the amount of pyrophosphate can be determined through measuring its amount produced accordingly. As a method of measuring the amount of D-ribitol-5-phosphate, a method in which increase of NADH (or NADPH) is calorimetrically determined through bringing it into the action of ribitol-5-phosphate dehydrogenase in the presence of NAD (or NADP).

As is described in above the first conventional technology, there are several kinds of discrimination techniques of the base type of a SNP site in which a primer extension reaction is utilized. In any one of discrimination techniques of the base type of a SNP site, it is common in respect that discrimination of the base type of a SNP site is executed by analyzing the difference of progress of the primer extension reactions.

There are two kinds of methods of analyzing the difference of primer extension reactions. One involves techniques in which an amplification method of a target base sequence such as PCR method, ICAN method, LCR method, SDA method, LAMP method or the like, and a technique for detecting a nucleic acid are utilized. In other words, amplification of a base sequence including a SNP site is carried out using the aforementioned typing primer as one primer. As a result, when the base at its 3′ end of the typing primer is complementary to a SNP site which is an object to be analyzed, a nucleic acid having a target base sequence can be well amplified, however, when it is not complementary thereto, the nucleic acid is hardly amplified. Therefore, by measuring the amount of the objective base sequence fragment with use of a labelling substance such as a fluorescent intercalating agent or the like, discrimination of the base type of a SNP site can be performed. However, as already stated, it is disadvantageous in the need of a very dangerous operation because the fluorescent intercalating agent is a carcinogen.

Another involves techniques in which an amplification method of a target base sequence such as PCR method, ICAN method, LCR method, SDA method, LAMP method or the like, and a technique for detecting pyrophosphate are utilized. In other words, although the same technique is applied to the amplification of a base sequence including a SNP site using the aforementioned typing primer as one primer. However, the nucleic acid is not detected in this method, but analysis of the amount of amplification of the target base sequence, i.e., discrimination of the base type of a SNP type, is carried out by detecting pyrophosphate that is produced with extension of the primer. In a known method of detecting pyrophosphate which may be used in this instance, pyrophosphate is converted to ATP, and thereafter a luciferase reaction is utilized. However, when dATP is used in a primer extension reaction, dATP becomes a substrate for the luciferase reaction similarly to ATP. Thus, accurate discrimination of the base type of a SNP site can not be achieved. Therefore, it is disadvantageous in that a special dATP analogue must be used which acts as a substrate for DNA polymerase instead of dATP, and does not act as a substrate for a luciferase reaction. In instances of this method, the base type of a SNP site can be also discriminated by using the typing primer alone and analyzing the primer extension reaction therefrom, differently from the method described above.

In addition, as described in the aforementioned second conventional technology, there exist disadvantages also in other technique for detecting pyrophosphate, in aspects that multiple kinds of enzymes, reagents and the like are needed, the cost is elevated, and the steps are complicated.

### SUMMARY OF THE INVENTION

The present invention was accomplished in order to solve the disadvantages as described hereinabove, and provides convenient technique for detecting an extension reaction of a primer, convenient techniques for discriminating the base type in a base sequence of a nucleic acid, and techniques for detecting a nucleic acid.

The method of detecting an extension reaction of the present invention in which an extension reaction of a primer is detected comprises: the step (a) of preparing a sample solution containing a nucleic acid, a primer having a base sequence that includes a complementary binding region which complementarily binds to the aforementioned nucleic acid, and a nucleotide; the step (b) of allowing the aforementioned sample solution to stand under a condition to cause the aforementioned extension reaction, and producing pyrophosphate when the aforementioned extension reaction is caused; the step (c) of bringing the aforementioned sample solution into contact with the front face of a H⁺ hardly permeable membrane having H⁺-pyrophosphatase, which penetrates from front to back of the membrane, of which active site that hydrolyzes pyrophosphate being exposed to the front face; the step (d) of measuring the H⁺ concentration of at least either one of the solution at the front face side of the aforementioned H⁺ hardly permeable membrane or the solution at the back face side of the aforementioned H+hardly permeable membrane, in a state where the aforementioned H⁺-pyrophosphatase is immersed in the solution; and the step (e) of detecting the aforementioned extension reaction on the basis of the result of measurement in the step (d).

The method of discriminating a base type of the present invention in which the base type in a base sequence of a nucleic acid is discriminated comprises: the step (a) of preparing a sample solution containing a nucleic acid, a primer having a base sequence that includes a complementary binding region which complementarily binds to the aforementioned nucleic acid, and a nucleotide; the step (b) of allowing the aforementioned sample solution to stand under a condition to cause an extension reaction of the aforementioned primer, and producing pyrophosphate when the aforementioned extension reaction is caused; the step (c) of bringing the aforementioned sample solution into contact with the front face of a H⁺ hardly permeable membrane having H⁺-pyrophosphatase, which penetrates from front to back of the membrane, of which active site that hydrolyzes pyrophosphate being exposed to the front face; the step (d) of measuring the H⁺ concentration of at least either one of the solution at the front face side of the aforementioned H⁺ hardly permeable membrane or the solution at the back face side of the aforementioned H⁺ hardly permeable membrane, in a state where the aforementioned H⁺-pyrophosphatase is immersed in the solution; the step (e) of detecting the aforementioned extension reaction on the basis of the result of measurement in the step (d); and the step (f) of discriminating the base type in the base sequence of the aforementioned nucleic acid on the basis of the result of detection in the step (e).

As a method of discriminating the base type in a base sequence of a nucleic acid, there exists, for example, a method in which the base type of a base to which the discrimination is intended is discriminated on the basis of the extent of progress of a primer extension reaction, when the primer extension reaction is carried out using a primer having a completely complementary sequence to the base sequence adjacent to the 3′ end side from the base to which the discrimination is intended, and dNTP which is complementary to the predicted base type of the base to which the discrimination is intended. Further, there also exists a method in which a primer is used that has a complementary base sequence to a base sequence including a base to which the discrimination is intended, and which causes a difference of the extent of the progress of a primer extension reaction depending on the base type of a base to which the discrimination is intended when the primer extension reaction is carried out using 4 kinds of dNTPs simultaneously. In any of these methods, it is common in respect that discrimination of the base type of a particular base is executed on the basis of the extent of the progress of the primer extension reactions. When a primer extension reaction is caused, pyrophosphate is produced. According to the present invention, the extent of the progress of the primer extension reactions can be analyzed through detecting pyrophosphate produced by primer extension reactions. Therefore, discrimination of the base type in a base sequence of a nucleic acid is permitted. The term “discrimination of the base type in a base sequence of a nucleic acid” herein refers to for example, discrimination as to whether or not a SNP site in a DNA is a particular base, determination of the base type of a SNP site, discrimination of the presence/absence of a mutation site, determination of a mutation site, and determination of the base type of a mutation site.

In natural world, H⁺-pyrophosphatase is retained in a tonoplast membrane such that the active site thereof which hydrolyzes pyrophosphate is exposed to outside of the tonoplast membrane (front face side), and it has a property to transport H⁺ from outside of the tonoplast membranes toward inside of the tonoplast membranes (back face side) accompanied by a hydrolysis reaction in which two molecules of phosphoric acid are formed from one molecule of pyrophosphate. Hence, the H⁺ concentration is increased within the tonoplast membrane due to the enzyme reaction of H⁺-pyrophosphatase, while the H⁺ concentration is decreased outside of the tonoplast membrane. According to the present invention, through reserving a sample solution, which is going to include pyrophosphate when an extension reaction proceeds, in a first region where an active site of H⁺-pyrophosphatase that hydrolyzes pyrophosphate is exposed, H⁺ is transported from the first region to a second region when the extension reaction proceeds, and thus the H⁺ concentrations at the front face side and the back face side of the tonoplast membrane vary. Consequently, the amount of pyrophosphate in the sample solution can be detected by measuring the H⁺ concentration at either one of the front face side or the back face side. therefore, according to the method of the present invention in which the base type in a base sequence of a nucleic acid is discriminated by detecting pyrophosphate produced by the extension reaction of the primer, multiple kinds of enzymes, reagents and the like for detecting pyrophosphate are not required, with simple steps, leading to reduction of the cost.

For example, in the step (d), the difference between the H⁺ concentration of the solution at the front face side, and the H⁺ concentration of the aforementioned sample solution post the step (b) and before the step (c) is measured. Additionally, in the step (e), the aforementioned extension reaction is detected by comparing the result of measurement in the step (d) with a control value. When the aforementioned discrimination of the base type is the discrimination of the base type of a SNP site, the control value described above can be the result of measurement obtained in the step (d) through carrying out the steps (a), (b), (c) and (d) using a nucleic acid having the aforementioned SNP site without mutation, as the nucleic acid described above.

Furthermore, the aforementioned extension reaction can be detected by, for example,detecting the H⁺ concentration of the solution at the back face side, in the step (d), and comparing the result of measurement in the step (d) with a control value, in the step (e). When the aforementioned discrimination of a base type is the discrimination of the base type of a SNP site, one kind of a nucleotide is used as the aforementioned nucleotide in the step (a), and the aforementioned control value can be the result of measurement obtained in the step (d) through carrying out the steps (a), (b), (c) and (d) using a nucleic acid having the aforementioned SNP site with a different base type, as the nucleic acid described above.

In the step (d), the concentration of H⁺ may be optically measured. In this instance, for example, the H⁺ concentration can be measured by adding a pH sensitive pigment or a membrane potential sensitive pigment to at least either one of the solution at the aforementioned front face side and the solution at the back face side, and measuring an optical response of the aforementioned pigment. Exemplary pH sensitive pigment described above includes e.g., acridine orange. Exemplary membrane potential sensitive pigment described above includes e.g., Oxol V.

In the step (d), the H⁺ concentration may be electrically measured.

The extension reaction may be for example, an extension reaction according to a PCR method.

The device for discriminating a base type in which the base type in a base sequence of a nucleic acid is discriminated of the present invention comprises a reaction section in which thermoregulation required for an extension reaction of a primer is carried out, and a pyrophosphate detection section in which pyrophosphate that is produced upon the aforementioned primer extension reaction is detected, wherein the aforementioned reaction section is provided with a reserving region for reaction where a solution is reserved, and the aforementioned pyrophosphate detection section is provided with a reserving region for detection where a solution is reserved, a H⁺ hardly permeable membrane that separates the aforementioned reserving region for detection to a first region and a second region, and a measurement means for measuring the H⁺ concentration of the solution reserved in at least either one of the first region and second region, wherein the aforementioned H⁺ hardly permeable membrane has H⁺-pyrophosphatase, which penetrates from front to back of the membrane, of which active site that hydrolyzes pyrophosphate being exposed to the front face, and in the aforementioned pyrophosphate detection section, the reaction solution which is delivered from the aforementioned reaction section is reserved in the first region.

As a method of discriminating the base type of a particular base there exists a, for example,method in which the base type of a base to which the discrimination is intended is discriminated on the basis of the extent of progress of a primer extension reaction, when the primer extension reaction is carried out using a primer having a completely complementary sequence to the base sequence adjacent to the 3′ end side from the base to which the discrimination is intended, and dNTP which is complementary to the predicted base type of the base to which the discrimination is intended. Further, there also exists a method in which a primer is used which has a complementary base sequence to a base sequence including a base to which the discrimination is intended, and which causes a difference of the extent of the progress of a primer extension reaction depending on the base type of a base to which the discrimination is intended when the primer extension reaction is carried out using 4 kinds of dNTPs simultaneously. In any of these methods, it is common in respect that discrimination of the base type of a particular base is carried out on the basis of the extent of the progress of the primer extension reactions. When a primer extension reaction is caused, pyrophosphate is produced. According to the device for discriminating a base type of the present invention, the extent of the progress of the primer extension reactions can be analyzed through measuring pyrophosphate produced by a primer extension reaction. Therefore, discrimination of the base type of a particular base is permitted.

Moreover, when discrimination of the presence/absence of a nucleic acid having a particular base sequence in a sample solution is intended, presence of a nucleic acid having a base sequence that is complementary to the primer in the solution is revealed, when the primer extension reaction proceeds. To the contrary, absence of a nucleic acid having a base sequence that is complementary to the primer in the solution is revealed, when the primer extension reaction does not proceed so much. Thus, using the device for discriminating a base type of the present invention, discrimination of the presence/absence of a nucleic acid having a particular base sequence in a sample solution, i.e., detection of a particular nucleic acid is also enabled.

The aforementioned measurement means can be constituted such that, for example, the H⁺ concentration is optically measured. In addition, the aforementioned measurement means can be constituted such that, for example, the H⁺ concentration is electrically measured.

The aforementioned device for discriminating a base type may further comprises an analysis means for controlling the aforementioned reaction section and the aforementioned pyrophosphate detection section, and for analyzing the result of measurement from the aforementioned measurement means.

The aforementioned device for discriminating a base type may further comprise a slot to which a tip can be inserted that is provided with the aforementioned reserving region for reaction and the aforementioned reserving region for detection.

The device for detecting pyrophosphate of the present invention comprises a vessel, a H⁺ hardly permeable membrane that separates inside of the aforementioned vessel into a first region and a second region, an electrode that is provided such that it is brought into contact with a solution reserved in the first region, and a H⁺ sensitive electrode that is provided such that it is brought into contact with a solution reserved in the second region, wherein the aforementioned H⁺ hardly permeable membrane has H⁺-pyrophosphatase which is arranged such that it penetrates from front to back of the membrane, and that the active site thereof that hydrolyzes pyrophosphate is exposed to the first region.

The method of detecting a nucleic acid having a particular base sequence of the present invention comprises: the step (a) of preparing a sample solution containing a sample, a primer having a base sequence that includes a complementary binding region which complementarily binds to the aforementioned nucleic acid, and a nucleotide; the step (b) of allowing the aforementioned sample solution to stand under a condition to cause an extension reaction of the aforementioned primer, and producing pyrophosphate when the aforementioned extension reaction is caused; the step (c) of bringing the aforementioned sample solution into contact with the front face of a H⁺ hardly permeable membrane having H⁺-pyrophosphatase, which penetrates from front to back of the membrane, of which active site that hydrolyzes pyrophosphate being exposed to the front face; the step (d) of measuring the H⁺ concentration of at least either one of the solution at the front face side of the aforementioned H⁺ hardly permeable membrane or the solution at the back face side of the aforementioned H⁺ hardly permeable membrane, in a state where the aforementioned H⁺-pyrophosphatase is immersed in the solution; the step (e) of detecting the aforementioned extension reaction on the basis of the result of measurement in the step (d); and the step (f) of detecting the nucleic acid on the basis of the result of detection in the step (e).

A primer hybridizes to a nucleic acid having a complementary base sequence, and is extended by a primer extension reaction. When a primer extension reaction is caused, pyrophosphate is produced. According to the present invention, the extent of progress of the primer extension reaction can be analyzed by detecting the amount of pyrophosphate, more specifically, by measuring the H⁺ concentration. When the primer extension reaction proceeded, it is revealed that a nucleic acid having a base sequence that is complementary to the primer is present in the sample solution. To the contrary, when the primer extension reaction did not proceed so much, it is revealed that a nucleic acid having a base sequence that is complementary to the primer is almost absent in the sample solution. In such a manner, the presence/absence of a nucleic acid having a particular base sequence in a sample solution can be discriminated.

For example, in the step (d), the difference between the H⁺ concentration of the solution at the front face side, and the H⁺ concentration of the aforementioned sample solution post the step (b) and before the step (c) is measured. Additionally, in the step (e), for example, the aforementioned extension reaction is detected by comparing the result of measurement in the step (d) with a control value. In this step, as the aforementioned control value, the result of measurement obtained in the step (d) through carrying out the steps (a), (b), (c) and (d) using the aforementioned sample without including a nucleic acid can be used.

In the step (d), the H⁺ concentration of may be optically measured. In this instance, the H⁺ concentration can be measured by, for example, adding a pH sensitive pigment or a membrane potential sensitive pigment to at least either one of the solution at the front face side and the solution at the back face side, and measuring an optical response of the aforementioned pigment. Exemplary pH sensitive pigment described above includes e.g., acridine orange. Exemplary membrane potential sensitive pigment described above includes e.g., Oxol V.

Also, the H⁺ concentration may be electrically measured in the step (d).

The aforementioned extension reaction maybe for example, an extension reaction according to a PCR method.

According to the tip for introducing a sample solution according to the present invention, it is constituted to provide a reaction chamber for carrying out an extension reaction of a primer, a pyrophosphate detection chamber for detecting pyrophosphate, and a flow pass that connects the aforementioned reaction chamber and the aforementioned pyrophosphate detection chamber.

In addition, the aforementioned flow pass may be constituted such that it can be opened and closed. In this instance, the reaction chamber and the pyrophosphate detection chamber can be readily separated. Therefore, the primer extension reaction and detection of pyrophosphate of which reaction temperature conditions are different with each other can be executed on one tip.

It is preferred that: the pyrophosphate detection chamber described above has a first region and a second region which are separated by a H⁺ hardly permeable membrane; the aforementioned H⁺ hardly permeable membrane has H⁺-pyrophosphatase which is arranged such that it penetrates from front to back of the membrane, and that the active site thereof that hydrolyzes pyrophosphate is exposed to the first region; and in the aforementioned pyrophosphate detection chamber, the reaction solution that is delivered from the aforementioned reaction chamber via the aforementioned flow pass is reserved in the first region.

When a sample solution is injected into the pyrophosphate detection chamber, an enzyme reaction of H⁺-pyrophosphatase is caused when pyrophosphate is present in the sample solution, thereby leading to increase of the H⁺ concentration in the second region which is separated by the membrane, and to decrease of the H⁺ concentration in the second region. Accordingly, the H⁺ concentration can be electrically measured with an electrode and a H⁺ sensitive electrode, thereby enabling the detection of the amount of pyrophosphate.

Object as described above, other objects, characteristics, and advantages of the present invention will be apparent from the following detailed description of the preferred embodiments with reference to the accompanying drawings.

### BRIEF DESCRIPTION OF THE DRAWINGS

FIG. 1 is a process drawing showing the method of discriminating the base type of a SNP site of a target DNA in a sample according to Embodiment 1.

FIG. 2 is a process drawing showing the method of discriminating the base type of a SNP site of a target DNA in a sample according to Embodiment 1.

FIG. 3 is a drawing schematically showing H⁺-pyrophosphatase.

FIG. 4 is a drawing showing a method of detecting pyrophosphate.

FIG. 5 is a drawing showing a device for detecting pyrophosphate.

FIG. 6 is a schematic drawing showing the device for discriminating a base type according to Embodiment 1.

FIG. 7 (a) is a top view schematically showing the tip according to Embodiment 1; and FIG. 7 (b) is a cross sectional view along the line X-X depicted in FIG. 7.

FIG. 8 is a top view schematically showing another tip according to Embodiment 1.

FIG. 9 is a perspective view schematically showing still another tip according to Embodiment 1.

FIG. 10 is a process drawing showing the method of detecting as to whether or not a DNA having a particular base sequence is included in a sample according to Embodiment 2.

FIG. 11 is a graph showing the relationship between the concentration of sodium pyrophosphate and the change of fluorescence intensity at 540 nm.

FIG. 12 is a graph showing the relationship between the concentration of sodium pyrophosphate and the change of fluorescence intensity at 639 nm.

FIG. 13 is a graph showing the relationship between the concentration of sodium pyrophosphate and the pH value.

FIG. 14 (a) is a drawing showing two kinds of primer C and primer D which can completely hybridize to a particular base sequence of λDNA; FIG. 14 (b) is a Table presenting the composition of PCR reaction liquids G and H; and FIG. 14 (c) is a flow chart showing the reaction temperature condition under which the PCR reaction was carried out.

FIG. 15 (a) is a graph showing the percentage change of fluorescence intensity before and after mixing a H⁺-pyrophosphatase liquid to the PCR reaction liquids G and H, respectively; FIG. 15 (b) is a formula indicating the percentage change of fluorescence intensity.

FIG. 16 (a) is a drawing showing a wild type λDNA, a mutant λDNA and a typing primer; FIG. 16 (b) is a Table presenting the composition of PCR reaction liquids I and J; and FIG. 16 (c) is a flow chart showing the reaction temperature condition under which the PCR reaction was carried out.

FIG. 17 illustrates the percentage change of fluorescence intensity before and after mixing the PCR reaction liquids I and J, respectively.

FIG. 18 (a) is a drawing showing a primer; FIG. 18 (b) is a Table presenting the composition of PCR reaction liquids K and L; and FIG. 18 (c) is a flow chart showing the reaction temperature condition under which the PCR reaction was carried out.

FIG. 19 is a process drawing showing the discrimination technique of the base type of a SNP site in which a conventional primer extension reaction is utilized.

FIG. 20 is a process drawing showing the discrimination technique of the base type of a SNP site in which a conventional primer extension reaction is utilized.

FIG. 21 is a process drawing showing the discrimination technique of the base type of a SNP site in which a conventional primer extension reaction is utilized.

FIG. 22 is a process drawing showing the discrimination technique of the base type of a SNP site in which a conventional primer extension reaction is utilized.

### DETAILED DESCRIPTION OF THE INVENTION

Embodiments of the present invention are explained below with reference to the drawings. Nucleic acids such as DNA and RNA described herein are double stranded unless specifically indicated.

### EMBODIMENT 1

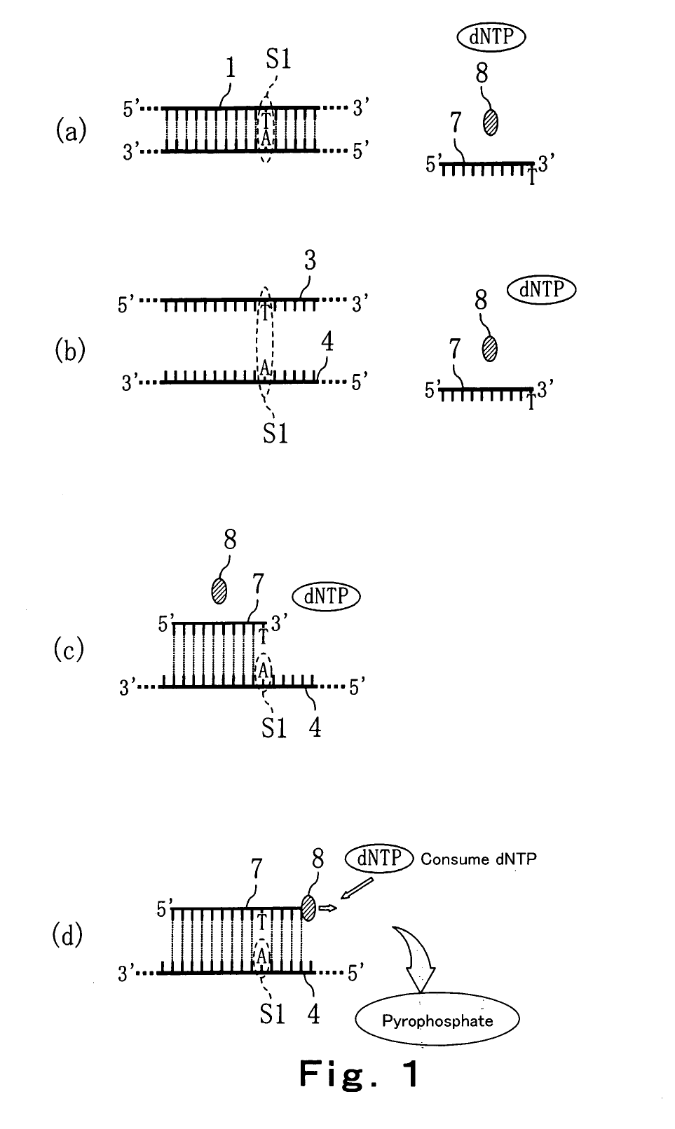

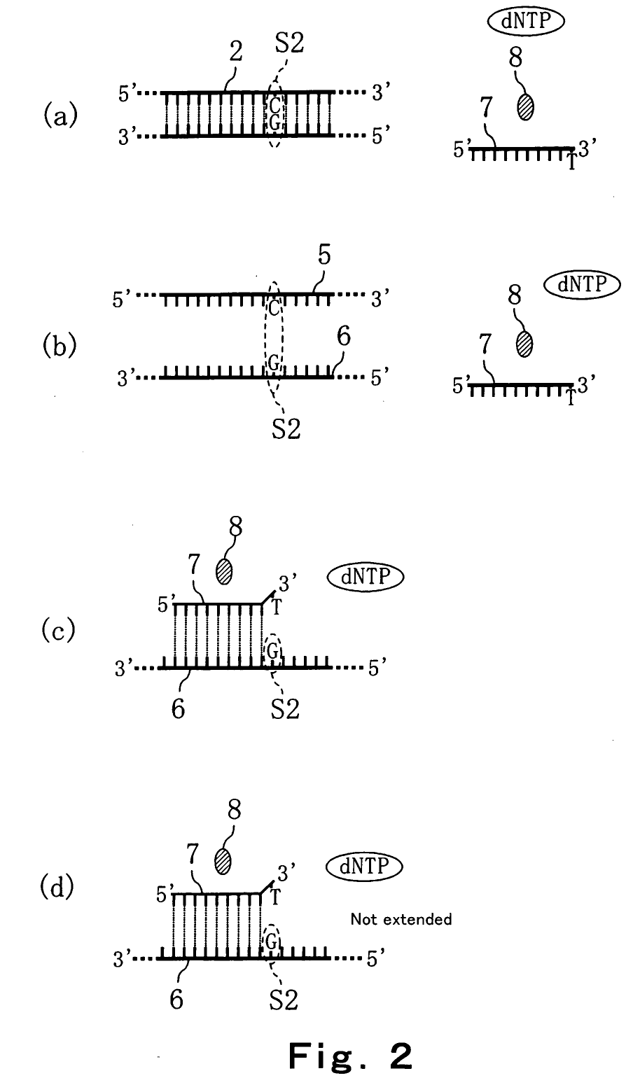

In this Embodiment, a method of discriminating the base type of a SNP site of a target DNA in a sample is explained. Specifically, methods in which a primer extension reaction (for example, an amplification reaction such as PCR method, ICAN method, LCR method, SDA method, LAMP method or the like) is utilized using 4 kinds of dNTPs is explained with reference to FIG. 1 and FIG. 2. FIG. 1 and FIG. 2 are process drawings showing the method of discriminating the base type of a SNP site of a target DNA in a sample according to this Embodiment.

In the method of this Embodiment, a primer is used which substantially complementarily binds to a base sequence including a SNP site of a target DNA, and causes the difference of progress of the extension reaction depending on the base type of the SNP site of the target DNA (hereinafter, referred to as a typing primer). In this Embodiment, an example is demonstrated in which there exists a possibility that the base of a SNP site in a single stranded target DNA on which a typing primer acts is A or G, and the typing primer for use is designed such that the primer extension reaction is not caused when the base is G, but is caused when the base is A.

First, in the step illustrated in FIG. 1 (a), a sample solution containing a target DNA 1 having a SNP site S1, a typing primer 7, DNA polymerase 8 and 4 kinds of dNTPs is prepared. Similarly, in the step illustrated in FIG. 2 (a), a sample solution containing a target DNA 2 having a SNP site S2, a typing primer 7, DNA polymerase 8 and 4 kinds of dNTPs. The typing primer 7 herein is designed such that it completely hybridizes to the 3′ end sided region from the SNP site of single stranded DNAs 4 and 6, except for its base at 3′ end (in this case, thymine (hereinafter, denoted as T)). Moreover, the DNA polymerase 8 used in this Embodiment is a known enzyme having heat resistance, which is generally used in PCR and the like.

Next, in the step illustrated in FIG. 1 (b), the sample solution is heated to subject the DNA 1 to thermal denaturation to give single stranded DNAs 3 and 4. Similarly, in the step illustrated in FIG. 2 (b), the sample solution is heated to subject the DNA 2 to thermal denaturation to give single stranded DNAs 5 and 6.

Next in the step illustrated in FIG. 1 (c), the sample solution is cooled to allow hybridization of the single stranded DNA 4 to the typing primer 7. Because the SNP site S1 of the single stranded DNA 4 is adenine (hereinafter, denoted as A), the typing primer 7 completely hybridizes to the single stranded DNA4. Similarly, in the step illustrated in FIG. 2 (c), the sample solution is cooled to allow hybridization of the single stranded DNA 6 to the typing primer 7. Because the SNP site S2 of the single stranded DNA 6 is guanine (hereinafter, denoted as G), the typing primer 7 does not hybridize to the single stranded DNA 4 only at its 3′ end base (T).

Next, in the step illustrated in FIG. 1 (d), the temperature of the sample solution is regulated to an optimal temperature for the primer extension reaction. The typing primer 7 has completely hybridized to the single stranded DNA 4. Thus, the primer extension reaction is caused, and dNTP is consumed by the DNA polymerase 8 to produce pyrophosphate.

On the other hand, also in the step illustrated in FIG. 2 (d), the temperature of the sample solution is regulated to an optimal temperature for the primer extension reaction. However, the typing primer 7 has been in a state where its 3′ end base (T) does not hybridize to the single stranded DNA 6. Thus, normal primer extension reaction hardly occurs. Therefore, dNTP is hardly consumed, and thus pyrophosphate is hardly produced.

Subsequently, by repeating the steps illustrated in FIG. 1 (b) to (d), and the steps illustrated in FIG. 2 (b) to (d) as described above, the primer extension reaction is repeated. Accordingly, the difference of progress of the primer extension reactions illustrated in FIG. 1 (d) and FIG. 2 (d) is remarkably exhibited.

Instead of the method of this Embodiment, a PCR reaction may be carried out using the typing primer 7 as one of two primers used in the PCR reaction, together with use of another primer. The difference of progress of the primer extension reactions is thereby expanded exponentially. In addition, other amplification reaction except for the PCR method can be also applied.

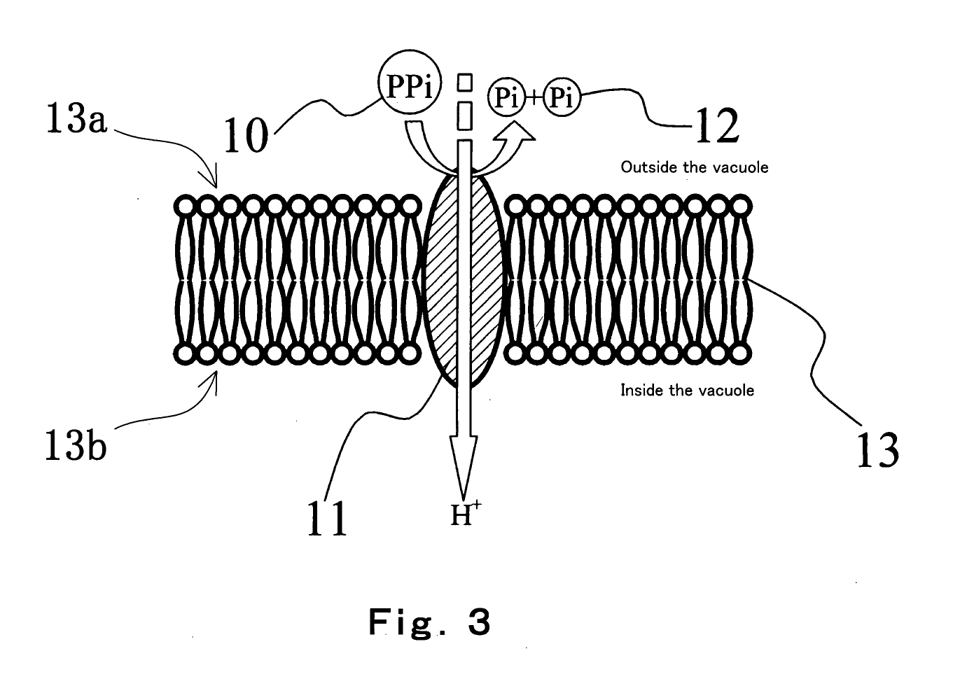

Finally, the difference of progress of the primer extension reactions illustrated in FIG. 1 (d) and FIG. 2 (d) is analyzed by quantitative detection of pyrophosphate. Accordingly, the discrimination of the base type of a SNP site is enabled. Next, the method of quantitatively detecting pyrophosphate of this Embodiment is explained with reference to FIG. 3.

In this Embodiment, H⁺-pyrophosphatase is used for detecting pyrophosphate. H⁺-pyrophosphatase is a membrane protein that generally exists in tonoplast membranes and the like of a plant. FIG. 3 is a drawing schematically showing H⁺-pyrophosphatase in a state indwelling in a tonoplast membrane of a plant.

As is shown in FIG. 3, H⁺-pyrophosphatase 11 has a property to transport H⁺ from the outside (front face 13a side) of the tonoplast membrane 13 that does not pass or hardly passes H⁺, toward the inside (back face 13b side) of the tonoplast membrane, accompanied by a hydrolysis reaction that produces two molecules of phosphoric acid 12 from one molecule of pyrophosphate 10. Thus, by the enzyme reaction of H⁺-pyrophosphatase, the H⁺ concentration is increased within the tonoplast membrane, while the H⁺ concentration is decreased outside of the tonoplast membrane.

In this Embodiment, utilizing the property of H⁺-pyrophosphatase as described above and its morphology, that is, a membrane protein, detection of pyrophosphate is effected. More specifically, the amount of pyrophosphate of which hydrolysis was responsible for H⁺-pyrophosphatase can be detected through separating a region by a membrane that retains H⁺-pyrophosphatase, and measuring the change of H⁺ concentration in at least either one region. Thus, detection of pyrophosphate is executed by detecting the change of the concentration of H⁺ directly participated in the action of H⁺-pyrophosphatase according to the method of this Embodiment, therefore, convenient detection with high sensitivity is enabled. Further, for executing the detection as described above, separation between the initiating region of the transportation of H⁺ and the receiving region of the transport of H⁺ becomes a prerequisite. However, the morphology of H⁺-pyrophosphatase can be utilized for such separation because it is a membrane protein. This contributes to the simplification of detection.

In this Embodiment, a sample solution containing pyrophosphate is brought into contact with H⁺-pyrophosphatase in a state indwelling in a tonoplast membrane which had been isolated from a plant or the like. Thereafter, the change of the H⁺ concentration of either inside of the tonoplast membrane or outside of the tonoplast membrane is measured. Chang of the H⁺ concentration of either inside of the tonoplast membrane or outside of the tonoplast membrane is, as described later in Examples, correlative to the amount of pyrophosphate in the sample solution. Therefore, the amount of pyrophosphate in a sample solution is detected by measuring the change of the H⁺ concentration. A sample solution including a larger amount of pyrophosphate is a sample solution in which an extension reaction of a primer proceeded, whilst a sample solution including a smaller amount of pyrophosphate is a sample solution in which an extension reaction of a primer hardly proceeded. In brief, the difference of progress of the extension reactions of a primer discriminates the base type of a SNP site. For example, when the solutions shown in FIG. 1 (d) and FIG. 2 (d) are compared, the larger amount of pyrophosphate is detected in the case of FIG. 1 (d) than in the case of FIG. 2 (d). On the basis of this result, the SNP site S1 of DNA 4 is discriminated as the base A which is complementary to the base T at 3′ end of the primer 7. Further, the SNP site S2 of DNA 6 is discriminated as other than the base A which is complementary to the base T at 3′ end of the primer 7. Since the base of the SNP site had proven to be A or G, in this Embodiment, the SNP site S2 of DNA 6 site is determined as G.

The presence/absence of pyrophosphate is determined depending on whether or not the concentration of H⁺ reached to a predetermined value, and thus the presence/absence of progress of the primer extension reaction may be determined depending on the presence/absence of pyrophosphate. In this specification, the detection in which the presence/absence of pyrophosphate is determined depending on whether or not the change of the H⁺ concentration reached to a predetermined value is referred to as qualitative detection of pyrophosphate. To the contrary, the detection of the value of the amount of pyrophosphate (for example, concentration) is referred to as quantitative detection of pyrophosphate.

Discrimination of the presence/absence of progress of the extension reaction of a primer, i.e., discrimination as to whether or not a base of the SNP site included in a sample has complementary to the counterpart base in the employed primer by the qualitative detection of pyrophosphate is explained. In regard to the case showing in FIG. 1 (d), for example, it is discriminated that the primer extension reaction proceeded, thereby determined that the SNP site is the base A that is complementary to the base T at 3′ end of the primer employed. In regard to the case shown in FIG. 2 (d), it is discriminated that the primer extension reaction did not proceed, thereby discriminated that the SNP site is not the base A that is complementary to the base T at 3′ end of the primer employed. The existence of a possibility that the base of the SNP site is A or G has been revealed in this Embodiment, as described above. Therefore, when the base of the SNP site is discriminated as other than A in FIG. 2 (d), only remaining possibility is that the base is G. Accordingly, the base type of a SNP site is determined.

Further, even though the base type of a SNP site is not previously specified to two types as in this Embodiment, the base type of the SNP site can be finally determined through the discrimination of the presence/absence of the progress of the extension reaction of the primer as described above, by carrying out the operation, in which whether or not a base of the SNP site included in the sample has complementary to the counterpart base in the used primer is discriminated, using multiple kinds of primers.

The “discrimination of the base type in a base sequence of a nucleic acid” herein includes any one of the discrimination as to whether or not the base type of a SNP site is a particular base type, and the determination of the base type of a SNP site.

Exemplary method for measuring the change of the H⁺ concentration includes the method in which the change of the H⁺ concentration is measured after converting it into an optical change, and the method of electrical measurement. Examples of the method in which the change of the H⁺ concentration is measured after converting it into an optical change include methods in which a pH test paper, a pH sensitive pigment, a membrane potential sensitive pigment or the like is used. Examples of the method of electrical measurement include metal electrode methods (hydrogen electrode method, quinhydrone electrode method, antimony electrode method and the like), glass electrode methods, ISFET electrode methods, patch clamp methods, LAPS (Light-Addressable Potentiometric Sensor) methods and the like.

By using the aforementioned method of measuring the change of the H⁺ concentration, and the aforementioned reaction of H⁺-pyrophosphatase in combination, pyrophosphate in a sample solution can be measured after converting it into an optical signal or an electrical signal.

The method of measuring the change of the H⁺ concentration is not limited to the method of the measurement as described above, but may be any method capable of converting the change of the H⁺ concentration into an optical change or an electrical change, and capable of sensing the optical change or the electrical change.

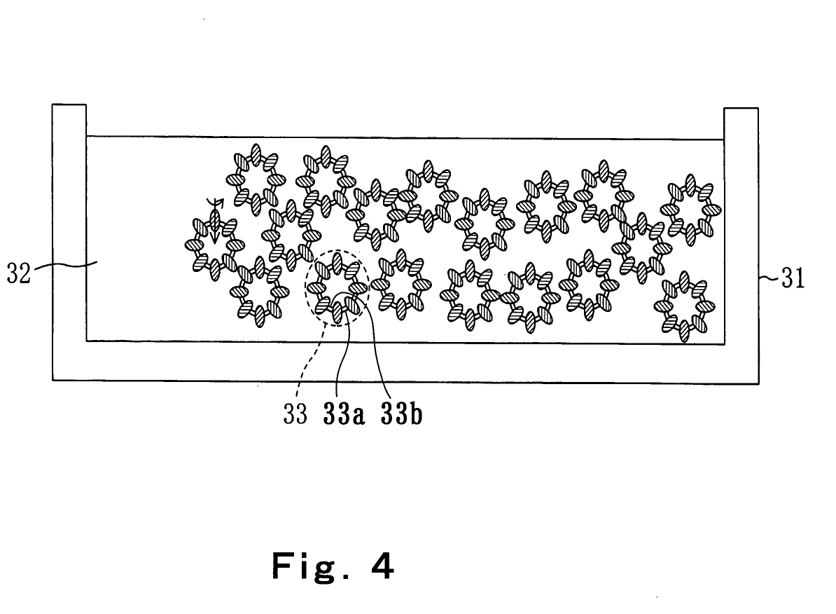

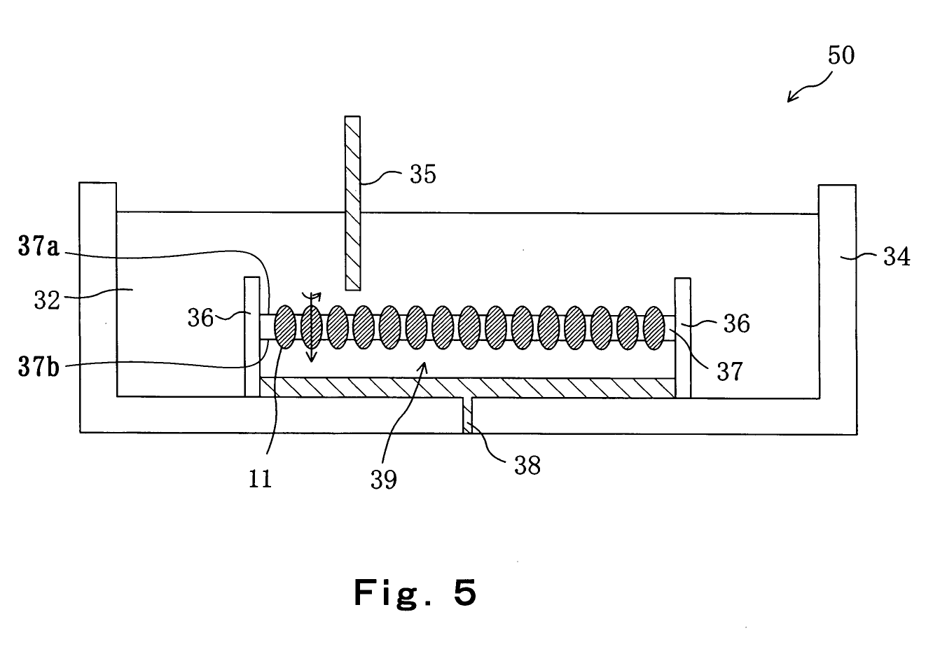

Next, the method of detecting pyrophosphate of this Embodiment is explained with reference to FIG. 4 and FIG. 5. FIG. 4 and FIG. 5 are drawings showing a method of detecting pyrophosphate.

As is shown in FIG. 4, a solution of suspended membrane vesicles 33 having H⁺-pyrophosphatase in dwelled in the membrane, and including a pH sensitive pigment or a membrane potential sensitive pigment therein is poured into a reaction vessel 31. Then, into the reaction vessel 31 is added a sample solution 32 obtained in FIG. 1 (d) or FIG. 2 (d). Upon this operation, the active site of H⁺-pyrophosphatase that hydrolyzes pyrophosphate is exposed to outside of the membrane vesicle (H⁺ hardly permeable membrane) 33. The solution included within the membrane vesicle 33 is not particularly limited as long as detection of the change of the H⁺ concentration by means of the transport of H⁺-pyrophosphatase is not inhibited. Herein, outer face 33a of the membrane vesicle 33 is referred to as a front face, while the inner face 33b is referred to as a back face. The pH sensitive pigment or membrane potential sensitive pigment may be added to the sample solution 32.

When pyrophosphate is present in the sample solution 32, an enzyme reaction of H⁺-pyrophosphatase is caused. Thus, the H⁺ concentration is increased inside of the membrane vesicle 33, while the H⁺ concentration is decreased outside of the membrane vesicle 33. Consequently, increase of the H⁺ concentration inside of the membrane vesicle 33 alters fluorescence intensity of the pH sensitive pigment or the membrane potential sensitive pigment. By optically measuring the change of this fluorescence intensity, qualitative detection and quantitative detection of pyrophosphate can be effected.

The membrane vesicle 33 which may be used is that prepared from vacuole isolated from cells. Further, as the membrane vesicle 33, any of those formed by isolating and purifying H⁺-pyrophosphatase, followed by reconstituting it within a membrane such as artificially formed lipid bilayer membrane, LB membrane or the like, which is not or hardly H⁺ permeable, such that the enzyme is in dwelled therein may be used.

H⁺-pyrophosphatase of which active site that hydrolyzes pyrophosphate is exposed inside maybe included in the membrane vesicle 33. However, when a membrane vesicle 33 including H⁺-pyrophosphatase of which active site that hydrolyzes pyrophosphate is exposed inside is used, it is preferred that the concentration of pyrophosphate inside of the membrane vesicle 33 is set to be lower than the concentration of pyrophosphate outside of the membrane vesicle. Most preferably, pyrophosphate is not included inside of the membrane vesicle 33. Thus, the transport of H⁺ from inside to outside of the membrane vesicle 33 decreases or arrests, thereby dominating the transport of H⁺ from outside to inside of the membrane vesicle 33. Accordingly, the change of the H⁺ concentration outside and inside of the membrane vesicle 33, is approximately limited to that resulting from pyrophosphate included in the sample solution 32. Therefore, the amount of pyrophosphate included in the sample solution 32 can be accurately estimated.

Moreover, membrane of the membrane vesicle 33 may include proteins other than H⁺-pyrophosphatase. However, these proteins are preferably proteins that do not react with pyrophosphate, or have low reactivity therewith. In other words, when pyrophosphate reacts with a protein, other than H⁺-pyrophosphatase, which is present in the membrane of the membrane vesicle 33, the amount of pyrophosphate which reacts with H⁺-pyrophosphatase is decreased, and concomitantly thereto, the amount of transport of H⁺ is decreased. In addition, when a protein which does not react with pyrophosphate, but which executes transport of H⁺ via a reaction with other substance than pyrophosphate is included in the membrane of the membrane vesicle 33, it is preferred that the substance with which the protein reacts is scarcely included in the sample solution 32. Specifically, when ATPase, a protein which hardly reacts with pyrophosphate, and which executes transport of H⁺ via a reaction with ATP, is included in the membrane of the membrane vesicle 33, it is preferred to render the sample solution 32 scarcely including ATP.

Exemplary pH sensitive pigment includes acridine orange. Further, exemplary membrane potential sensitive pigment includes Oxol V. Both of these are extremely sensitive pigments on a slight change of pH or membrane potential. Accordingly, detection of pyrophosphate with high sensitivity is enabled.

Also, a device for detecting pyrophosphate shown in FIG. 5 may be used. As shown in FIG. 5, a device for detecting pyrophosphate 50 is equipped with a vessel 34, an electrode 35, an internal chamber 36 provided within the vessel 34. In the internal chamber 36, is formed a membrane (H⁺ hardly permeable membrane) 37 having indwelling H⁺-pyrophosphatase, while the bottom of the inner chamber 36 is provided with a H⁺ sensitive electrode 38. The active site of H⁺-pyrophosphatase which hydrolyzes pyrophosphate is exposed outside of the internal chamber 36. The membrane 37 has its upper face 37a as a front face, and the lower face 37b as a back face.

Upon injection of a sample solution 32 into the vessel 34, when pyrophosphate is present in the sample solution 32, the enzyme reaction of H⁺-pyrophosphatase is caused. Hence, the H⁺ concentration of the solution in the internal region (second region) 39 of the internal chamber 36 separated with the membrane 37 is increased, whilst the H⁺ concentration outside of the internal chamber 36 is decreased. Thus, pyrophosphate can be qualitatively or quantitatively detected by electrically measuring the change of the H⁺ concentration using the electrode 35 and H⁺ sensitive electrode 38. The sample solution 32 is injected into the vessel 34 after reserving a solution, such as a buffer, which allows for the measurement of pH, in the vessel 34 and internal region 39 previously in this Embodiment, but not limited thereto. For example, the membrane 37 may be previously arranged on the H⁺ sensitive electrode 38 within the internal chamber 36, and the sample solution 32 may be then added to the vessel 34. By this operation, electrical measurement of the change of the H⁺ concentration is enabled using the electrode 35 and the H⁺ sensitive electrode 38, upon injection of the sample solution 32 into the vessel 34, thereby the internal region 39 filled with components that permeate the membrane 37 in the sample solution 32 (i.e., the solution without including pyrophosphate).

Additionally, H⁺-pyrophosphatase of which active site that hydrolyzes pyrophosphate is exposed to the internal region 39 may be included in the membrane 37. However, when the membrane 37 including H⁺-pyrophosphatase of which active site that hydrolyzes pyrophosphate is exposed to the internal region 39 is used, it is preferred that the concentration of pyrophosphate in the internal region 39 is set to be lower than the concentration of pyrophosphate outside of the internal chamber 36. Most preferably, pyrophosphate is not included within the internal region 39. Thus, transport of H⁺ from the internal region 39 to outside of the internal chamber 36 decreases or arrests, thereby dominating the transport of H⁺ from outside of the internal chamber 36 into the internal region 39. Accordingly, the change of the H⁺ concentration outside of the internal chamber 36 and in the internal region 39 is approximately limited to that resulting from pyrophosphate included in the sample solution 32. Therefore, the amount of pyrophosphate included in the sample solution 32 can be accurately estimated.

Moreover, the membrane 37 may include proteins other than H⁺-pyrophosphatase. However, these proteins are preferably proteins that do not react with pyrophosphate, or have low reactivity therewith. In other words, when pyrophosphate reacts with a protein, other than H⁺-pyrophosphatase, which is present in the membrane 37, the amount of pyrophosphate which reacts with H⁺-pyrophosphatase is decreased, and concomitantly thereto, the amount of transport of H⁺ is decreased. In addition, when a protein which does not react with pyrophosphate, but which executes transport of H⁺ via a reaction with other substance than pyrophosphate is included in the membrane 37, it is preferred that the substance with which the protein reacts is scarcely included in the sample solution 32. Specifically, when ATPase, a protein which hardly reacts with pyrophosphate, and which executes transport of H⁺ via a reaction with ATP, is included in the membrane 37, it is preferred to render the sample solution 32 scarcely including ATP.

Furthermore, the amount of pyrophosphate is electrically measured with the electrode 35 and the H⁺ sensitive electrode 38 in the device for detecting pyrophosphate 50, but not limited thereto. For example, a solution including a pH sensitive pigment or a membrane potential sensitive pigment may be added to the internal region 39 of the internal chamber. Fluorescence intensity of the pH sensitive pigment or the membrane potential sensitive pigment is thereby altered concomitantly with increase of the internal H⁺ concentration. The amount of pyrophosphate can be measured by optically measuring the alteration of this fluorescence intensity.

As described hereinabove, shape of the membrane with indwelling H⁺-pyrophosphatase for use in detecting pyrophosphate may be either spherical or planate. In other words, a condition may be just constituted in which all or almost all migration of H⁺ between two regions that are separated by the membrane with indwelling H⁺ -pyrophosphatase is effected by H⁺-pyrophosphatase.

In addition, discrimination of the presence/absence of a mutation site in a base sequence in a sample, determination of a mutation site, and determination of the base type of a mutation site can be carried out through utilizing the method of discriminating the base type of a SNP site of a target DNA in a sample of this Embodiment. In the discrimination of the presence/absence of a mutation site, the presence/absence of a mutation site in a base sequence in a sample is discriminated by causing a primer extension reaction using a primer that is completely complementary to an intended base sequence having no mutation site; and determining as to whether or not the amount of pyrophosphate produced by the reaction is nearly equal to or less than the amount of pyrophosphate produced (control value) when a primer extension reaction is caused using a sample containing an intended base sequence and a primer that is completely complementary to the intended base sequence. In other words, when it is discriminated that thus determined value is nearly equal to the standard value, a mutation site is concluded to be absent, whilst when it is discriminated that thus determined value is less than the standard value, a mutation site is concluded to be present.

In instances of determination of a mutation site, a mutation site can be determined by: causing a primer extension reaction using multiple primers designed to have each one base shifted; measuring the amount of thus produced pyrophosphate; and specifying the site corresponding the 3′ end of the primer that provides a minimum amount of pyrophosphate.

Determination of the base type of a mutation site can be carried out by a similar method of determining the base type of a SNP site as described above, after the determination of the mutation site.

The term “discrimination of the base type in a base sequence” of a nucleic acid herein includes any one of: discrimination of the presence/absence of a mutation site in a base sequence; determination of a mutation site; and determination of the base type of a mutation site.

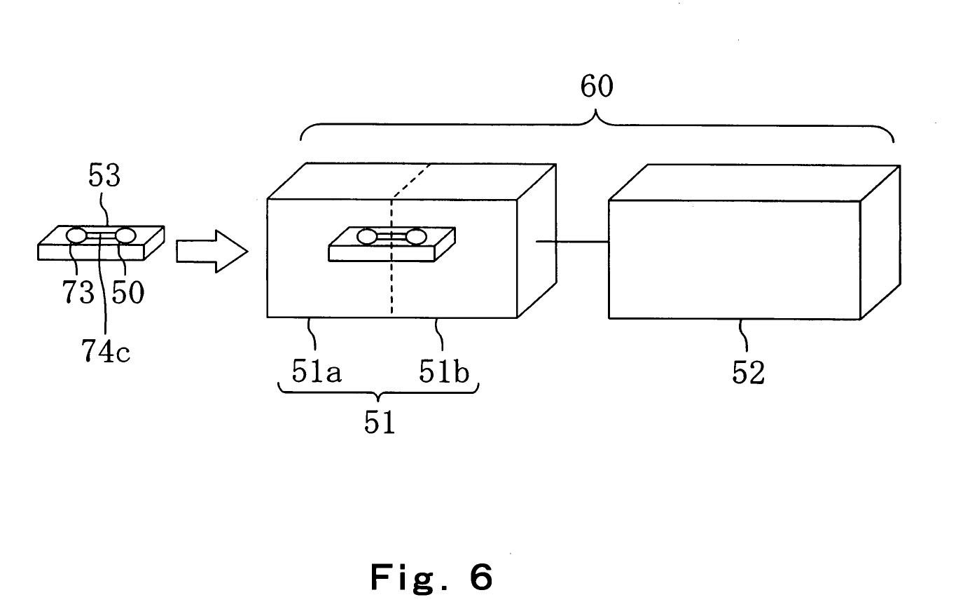

Next, the device for discriminating the base type of a SNP site of a target DNA in a sample is explained. FIG. 6 is a schematic drawing showing the device for discriminating a base type according to this Embodiment.

As is shown in FIG. 6, the device for discriminating a base type 60 has a reaction means 51 equipped with a reaction section 51a for executing a primer extension reaction and a pyrophosphate detection section 51b for detecting pyrophosphate; and an analysis means 52 for controlling the reaction section 51a and the pyrophosphate detection section 51b, and analyzing the obtained result. Further, the reaction means 51 has a slot to which a tip 53 can be inserted for introducing a sample solution.

The reaction section 51a may be constituted such that it enables thermoregulation which is required for the primer extension reaction. For example, when a PCR method is employed as the primer extension reaction, the reaction section 51a preferably has a constitution equipped with a heater section and a programmed thermal control section which can control the temperatures of the sample solution within the tip 53 for introducing the sample solution, to be suited for: denaturation of the nucleic acid; annealing of the primer; and the primer extension reaction by polymerase, respectively, for a predetermined time period. Additionally, when an isothermal reaction such as in an ICAN method, LAMP method and the like is employed, the reaction section 51a preferably has a constitution equipped with a heater section and a thermal control section capable of keeping a constant temperature (e.g., 65° C.). In this Embodiment, the same constitution as a thermal cycler for use in a PCR method is adopted.

The constitution of the pyrophosphate detection section 51b varies depending on the measurement means for measuring the change of the H⁺ concentration. In instances where the change of the H⁺ concentration is optically measured using a pigment such as a pH sensitive pigment or a membrane potential sensitive pigment as shown in FIG. 4 described above, the pyrophosphate detection section 51b preferably has a constitution equipped with a light source section for excitation of the fluorescent pigment, and a fluorescence intensity measurement section for measuring the intensity of the generated fluorescence.

Moreover, in instances where the change of the H⁺ concentration is electrically measured using an electrode as shown in FIG. 5described above, the pyrophosphate detection section 51b preferably has a constitution equipped with a contact section or a terminal that is capable of electrically connecting to the electrode 35 and the H⁺ sensitive electrode 38, respectively; and an electric potential difference measurement section capable of measuring the electric potential difference between the electrode 35 and the H⁺ sensitive electrode.

For introducing the sample solution, the tip 53 is equipped with a PCR chamber (a reserving region for reaction) 73, the device for detecting pyrophosphate (including a reserving region for detection) 50 shown in FIG. 5 as described above, a flow pass 74c that connects between the PCR chamber 73 and the device for detecting pyrophosphate 50.

The PCR chamber 73 is a chamber for carrying out PCR (primer extension reaction) in a sample solution containing a purified DNA, a typing primer, DNA polymerase and 4 kinds of dNTPs. In the PCR chamber 73 may be previously charged necessary reagents, respectively, or such reagents may be introduced immediately before inserting the tip into the device for discriminating a base type 60.

Since the device for detecting pyrophosphate 50 has the constitution as explained above, the explanation is now omitted. It is possible to use a device in which the change of the H⁺ concentration is converted to an optical change or an electrical change, and can sense thus resulting optical change or electrical change, instead of the device for detecting pyrophosphate 50.

The flow pass 74c is provided with a closing member. When the closing member is in its open state, flow of a fluid in the flow pass 74c is permitted, and when the closing member is in its closed state, the flow of a fluid in the flow pass 74c is blocked. By way of such a constitution, a structure with the PCR chamber 73 and the device for detecting pyrophosphate 50 being separated with each other is provided. The closing member is constituted such that opening and closing is allowed by the reaction means 51 of the device for discriminating a base type 60 described above. In the tip 53, the flow pass 74c does not necessarily have a constitution equipped with a closing member, as long as it is constituted such that: in the step of the PCR reaction, the reaction solution is retained in the PCR chamber 73 while inhibiting influx of a solution from outside; and in the step of detecting pyrophosphate, the solution post reaction is retained within the device for detecting pyrophosphate 50, while inhibiting influx of a solution from outside.

Furthermore, the analysis means 52 is connected to the reaction means 51, and maybe specifically, a personal computer (PC) or the like.

Operations of the device for discriminating a base type 60 are as follows.

First, a tip 53 with a PCR chamber 73 to which a sample solution is introduced containing a target DNA having a SNP site, a typing primer, DNA polymerase and 4 kinds of dNTPs is provided.

Next, the tip 53 is inserted into a slot of a reaction means 51. As shown in FIG. 6, when the tip 53 is inserted into the slot of the reaction means 51, the tip 53 is disposed within the reaction means 51 such that the PCR chamber 73 is positioned within the reaction section 51a (the PCR chamber 73 and the reaction section 51a are also collectively referred to as a reaction section), and that the device for detecting pyrophosphate 50 is positioned within the pyrophosphate detection section 51b (the device for detecting pyrophosphate 50 and the pyrophosphate detection section 51b are also collectively referred to as pyrophosphate detection section), respectively.

Next, the reaction means 51 renders a primer extension reaction caused in the sample solution which has been introduced into the PCR chamber of the tip 53 through repeating the steps illustrated in FIGS. 1 (b) to (d), and the steps illustrated in FIGS. 2 (b) to (d) in the reaction section 51a. Number of times of repeating the steps illustrated in FIGS. 1 (b) to (d), and the steps illustrated in FIGS. 2 (b) to (d) described above is set in the analysis means 52 beforehand.

Next, when the steps illustrated in FIGS. 1 (b) to (d) and the steps illustrated in FIGS. 2 (b) to (d) are completed, the flow pass 74c of the tip 53 is opened by the reaction means 51, and thus the sample solution is introduced into the device for detecting pyrophosphate 50. The pyrophosphate detection section 51b detects the amount of pyrophosphate generated by the primer extension reaction. Because specific method of detection is as described above, the explanation is now omitted.

Next, the analysis means 52 analyzes the result obtained from the pyrophosphate detection section 51b to discriminate the base type of the SNP site of the target DNA in the sample. Discrimination of the base type referred to herein includes any one of: discrimination as to whether or not it is a particular base type; and determination of the base type. In addition, using the device for discriminating a base type 60 illustrated in FIG. 6, discrimination of the presence/absence of a mutation site in a base sequence, determination of a mutation site, and determination of the base type of a mutation site can be also performed. In these instances, the result obtained from the pyrophosphate detection section 51b is analyzed in the analysis means 52, thereby conduting discrimination of the presence/absence of a mutation site in a base sequence, determination of a mutation site, and determination of the base type of a mutation site.

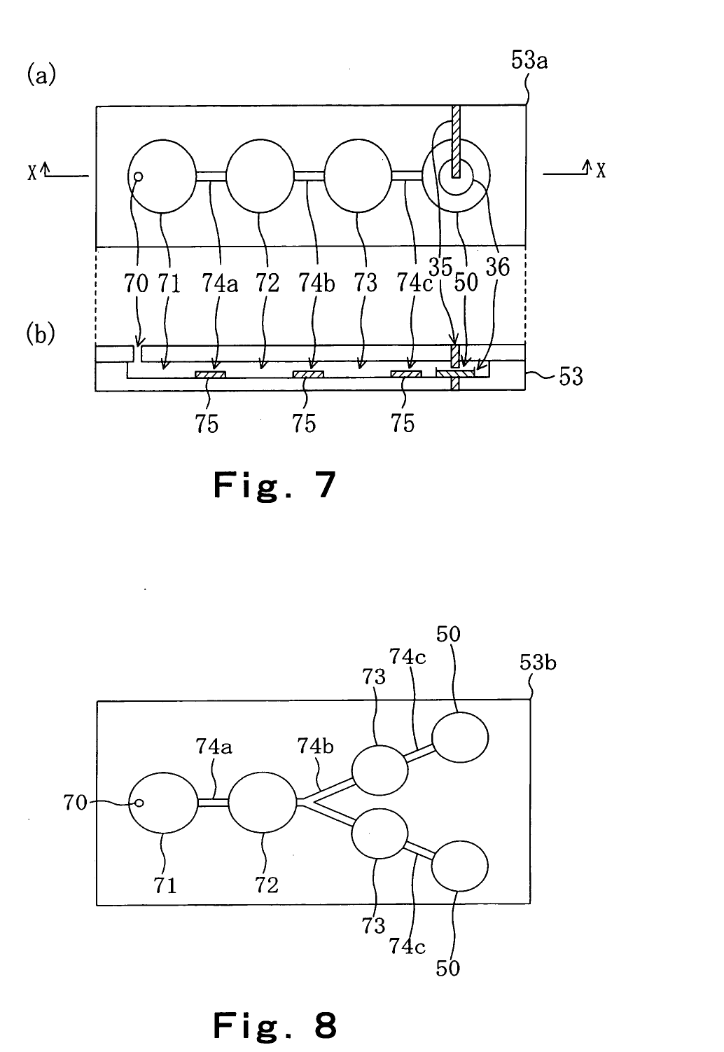

Next, another tip 53a which can be used instead of the tip 53 is explained. FIG. 7 (a) is a top view schematically showing another tip according to this Embodiment, and FIG. 7 (b) is a cross sectional view along the line X-X depicted in FIG. 7.

As shown in FIG. 7 (a) and (b), the tip 53a is equipped with a sample injection port 70, a DNA extraction chamber 71, a DNA purification chamber 72, a PCR chamber 73, a device for detecting pyrophosphate (including a reserving region for detection) 50, a flow pass 74a that connects the DNA extraction chamber 71 and the DNA purification chamber 72, a flow pass 74b that connects the DNA purification chamber 72 and the PCR chamber 73, and a flow pass 74 c that connects the PCR chamber (reserving region for reaction) 73 and the device for detecting pyrophosphate 50. In brief, the tip 53a has a constitution further comprising a sample injection port 70, a DNA extraction chamber 71, a DNA purification chamber 72, and flow passes 74a and 74b in addition to the tip 53 as illustrated in FIG. 6.

The sample injection port 70 links between outside and the DNA extraction chamber 71. A sample solution of a samlpe such as blood, saliva, hair, hair root or the like, which was subjected to a treatment with a drug solution as needed, is injected from the sample injection port 70 to the DNA extraction chamber 71.

The DNA purification chamber 72 is a chamber in which a treatment with a drug solution is carried out for purifying the DNA to eliminate impurities. As a matter of course, it may have a constitution that is provided with a column for purifying the DNA.

The PCR chamber73 is a chamber for carrying out PCR (primer extension reaction) in the sample solution containing the DNA purified in the DNA purification chamber 72, a typing primer, DNA polymerase and 4 kinds of dNTPs.

In the DNA extraction chamber 71, DNA purification chamber 72 and PCR chamber 73, may be previously charged necessary reagents, respectively, or such reagents may be introduced immediately before insertion into the device for discriminating a base type 60.

Since the device for detecting pyrophosphate 50 has the constitution as explained above, the explanation is now omitted. It is possible to use a device in which the change of the H⁺ concentration is converted to an optical change or an electrical change, and can sense thus resulting optical change or electrical change, instead of the device for detecting pyrophosphate 50.

The flow passes 74a, 74b and 74c are provided with a closing member 75. It has a structure to result in sealing of the DNA extraction chamber 71, the DNA purification chamber 72, the PCR chamber 73 and the device for detecting pyrophosphate 50, respectively, when each closing member 75 is lifted up. The closing member 75 is constituted such that opening and closing is allowed by the analysis means 52 of the device for discriminating a base type 60 described above.

Moreover, instead of the closing member 75, for example, a back flow prevention valve or the like may be disposed to the flow passes 74a, 74b and 74c. In addition, it may have such a constitution that the sample solution is transported to each section of the DNA extraction chamber 71, DNA purification chamber 72, PCR chamber 73 and device for detecting pyrophosphate 50, by: providing a de aerating opening that communicates to the device for detecting pyrophosphate 50; mounting an air discharge pump to the sample injection port 70; and mounting an air intake pump to the aforementioned de aerating opening. Further, the air discharge pump and the air intake pump described above may be constructed as a discharge and intake pump of oil that is non miscible to the sample solution. In every constitution, it is acceptable if separation of the DNA extraction chamber 71, the DNA purification chamber 72, the PCR chamber 73 and the device for detecting pyrophosphate 50, in the reaction means 51 of the aforementioned device for discriminating a base type 60, respectively, is possible. The term “separation” herein refers to a state in which a solution to be treated is retained in each chamber 71, 72, 73 during the treatment in each chamber71, 72, 73with preventing inflow of other solution. Therefore, the object can be achieved without providing a close member or the like as long as it is constituted such that each chamber 71, 72, 73 can be separated. For example, feasible constitution is that each chamber 71, 72, 73 is depressed lower than the flow pass 74a, 74b, 74c; and under the state in which solutions are kept in each chamber 71, 72, 73, the absence of inflow and outflow of the solutions is secured unless a liquid feeding means or the like is operated. Thus, the primer extension reaction and detection of pyrophosphate can be carried out on a single tip although they require different conditions of the enzyme reaction (e.g., optimal temperature and the like).

Additionally, the DNA extraction chamber 71, the DNA purification chamber 72 and the PCR chamber 73 are separated to individualized chambers in the tip 53a of this Embodiment, however, it may be constituted such that extraction of a DNA, purification of a DNA and PCR are carried out in a single chamber.

FIG. 8 is a top view schematically showing another tip according to this Embodiment.

As is shown in FIG. 8, the tip 53b is, similarly to the tip 53a shown in FIG. 7, equipped with a sample injection port 70, a DNA extraction chamber 71, a DNA purification chamber 72, a PCR chamber (reserving region for reaction) 73, a device for detecting pyrophosphate (including a reserving region for detection) 50, a flow pass 74a that connects between the DNA extraction chamber 71 and the DNA purification chamber 72, a flow pass 74b that connects between the DNA purification chamber 72 and the PCR chamber 73, and a flow pass 74 c that connects between the PCR chamber 73 and the device for detecting pyrophosphate 50. In particular, the flow pass 74b is bifurcated, and PCR chambers 73, devices for detecting pyrophosphate 50, and flow passes 74c that connect the PCR chamber 73 and the device for detecting pyrophosphate 50 are provided by two, respectively.

By introducing typing primers, which are different with each other, to the two PCR chambers 73 using the tip 53b, simultaneous discrimination of base types of two SNP sites is enabled. Further, two kinds of typing primers can be simultaneously introduced for a single SNP site, thereby resulting in usefulness in determination of the base type of a SNP site.

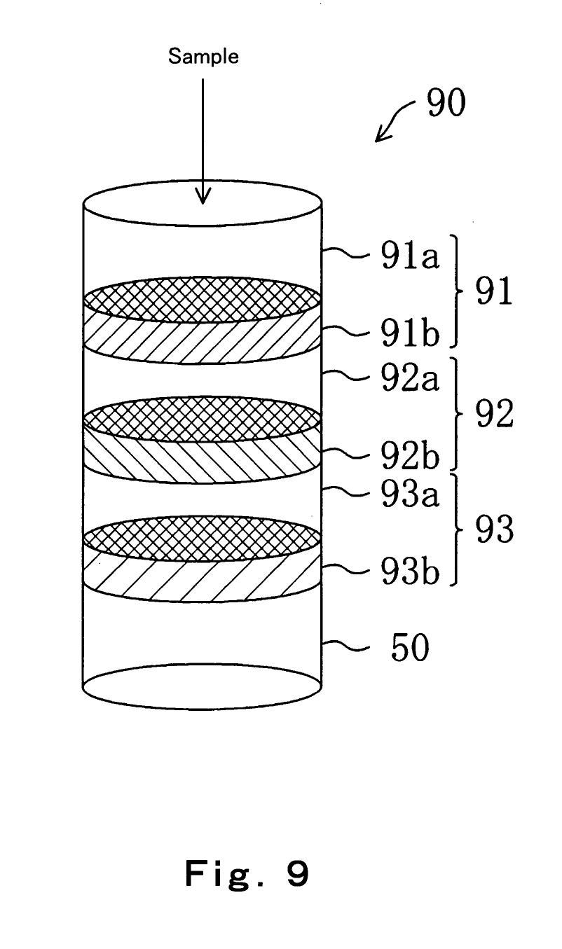

FIG. 9 is a perspective view schematically showing still another tip (vertical tip) of this Embodiment.

As shown in FIG. 9, the tip 90 is equipped with a sample introduction section 91, a DNA purification section 92, a PCR section 93 and a device for detecting pyrophosphate 50.

The sample introduction section 91 has a sample introduction chamber 91a and a DNA extraction column 91b. A sample solution of a sample such as blood, saliva, hair, hair root or the like, which was subjected to a treatment with a drug solution as needed, is injected to the sample introduction chamber 91a, and passes through the DNA extraction column 91b. A liquid such as blood, saliva or the like may be injected to the sample introduction chamber 91a without any treatment with a drug solution.

The DNA purification section 92 has a DNA purification chamber 92a and a DNA purification column 92b. The sample solution after passing through the DNA extraction column 91b is introduced to the DNA purification chamber 92a, and subsequently, passes through the DNA purification column 92b.

The PCR section 93 has a PCR chamber (reserving region for reaction) 93a and a separating member 93b. The sample solution including a DNA which was purified by passing through the DNA purification column 92b is introduced to the PCR chamber 93a, and thereto are added a DNA, a typing primer, DNA polymerase and 4 kinds of dNTPs. Accordingly, PCR (primer extension reaction) is caused.

The separating member 93b is constituted such that it can be opened and closed by the reaction means 51 of the aforementioned device for discriminating a base type 60. When PCR is terminated in the PCR chamber 93a, the reaction means 51 renders the separating member 93b open, thereby allowing passage of the sample solution into the device for detecting pyrophosphate (including a reserving region for detection) 50.

The tips 53a, 53b, 90 as described hereinabove are constituted to be equipped with a DNA purification chamber 72 or 92a, however, it is enough herein to merely perform the treatment of the sample solution such that any inhibitor of the PCR reaction is not included in the sample solution, or an inhibitor of the PCR reaction is inactivated.

Since the device for detecting pyrophosphate 50 has the constitution as explained above, the explanation is now omitted. It is possible to use a device in which the change of the H⁺ concentration is converted to an optical change or an electrical change, and can sense thus resulting optical change or electrical change, instead of the device for detecting pyrophosphate 50.

Although the difference of progress of the primer extension reactions is analyzed by detecting the amount of pyrophosphate in this Embodiment, as a matter of course, the amount of pyrophosphate that is present in a sample solution can be measured accurately, with no limitation to the primer extension reaction.

Further, in a primer extension reaction in particular, ATP and dATP become an inhibitor of H⁺-pyrophosphatase, therefore, when ATP or dATP is present in the sample solution, and the amount of pyrophosphate is small, the H⁺ concentration is scarcely changed. To the contrary, when dATP in the sample solution is consumed by the primer extension reaction, and the amount of pyrophosphate is large, the H⁺ concentration is greatly changed. In brief, the difference of progress of the primer extension reactions can be measured as a greater difference. Therefore, the base type can be discriminated with a higher degree of accuracy.

### EMBODIMENT 2

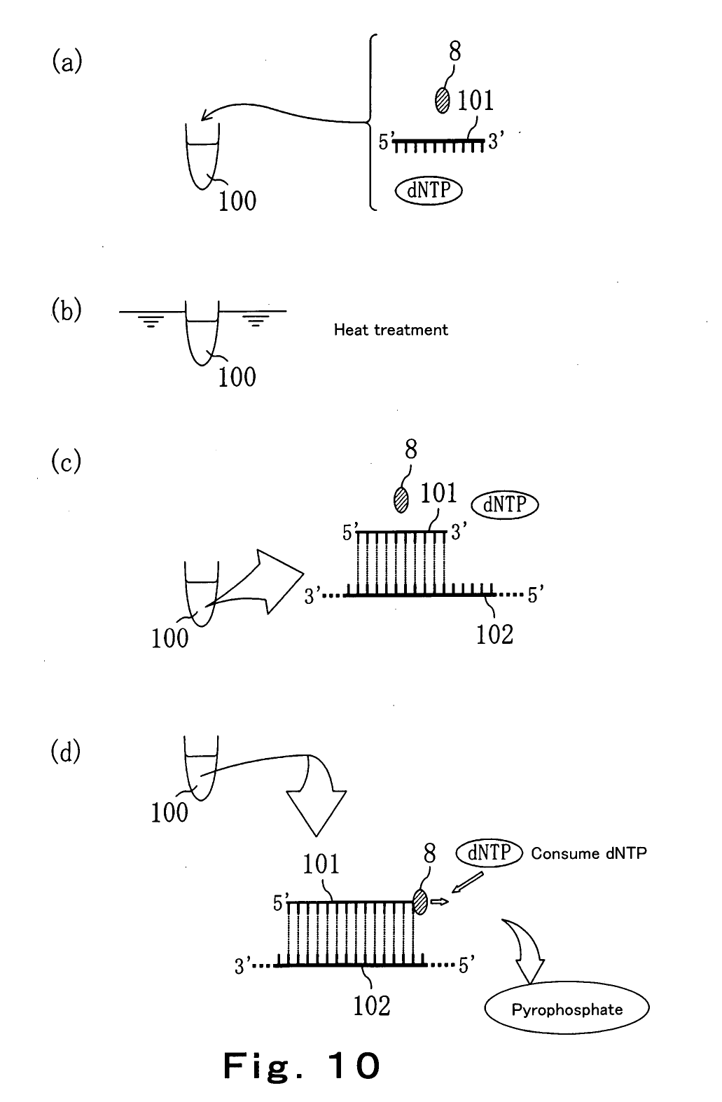

In this Embodiment, a method of discriminating as to whether or not a DNA having a particular base sequence is included in a sample, i.e., a method of detecting a DNA having a particular base sequence is explained. Specifically, methods in which a primer extension reaction (for example, an amplification reaction such as PCR method, ICAN method, LCR method, SDA method, LAMP method or the like) using 4 kinds of dNTPs is utilized is explained with reference to FIG. 10. FIG. 10 is a process drawing showing the method of discriminating as to whether or not a DNA having a particular base sequence is included in a sample according to this Embodiment.

In the method of this Embodiment, a primer having a base sequence which can complementarily bind to a DNA having a particular base sequence is used.

First, in the step illustrated in FIG. 10 (a), a primer 101 having a base sequence which can complementarily bind to a DNA having a particular base sequence, DNA polymerase 8 and 4 kinds of dNTPs are added to a solution to which discrimination is intended as to whether or not a DNA having a particular base sequence is included therein. Thus, a sample solution 100 is prepared. The primer 101 is designed such that it completely hybridizes to a single stranded DNA having the particular base sequence.

Next, in the step illustrated in FIG. 10 (b), a heat treatment of the sample solution 100 is carried out. Almost all DNA included in the sample solution 100 is thereby converted to the single stranded DNA.

Next in the step illustrated in FIG. 10 (c), the sample solution 100 is cooled. Accordingly, when a single stranded DNA 102 produced from the DNA having the particular base sequence is present in the sample solution 100, the primer 101 is hybridized to the single stranded DNA 102.

Next, in the step illustrated in FIG. 10 (d), the temperature of the sample solution 100 is regulated to an optimal temperature for the primer extension reaction. When the single stranded DNA 102 is present, the primer 101 is hybridized to the single stranded DNA 102, resulting in the primer extension reaction. Accordingly, dNTP is consumed by the DNA polymerase 8, thereby producing pyrophosphate.

In this step, when a single stranded DNA 102 having the particular base sequence is not present, the primer 101 can not achieve hybridization. Hence, the primer extension reaction does not occur. Therefore, dNTP is scarcely consumed, and pyrophosphate is hardly produced.

Next, the presence/absence of progress of the primer extension reaction is discriminated by qualitatively detecting pyrophosphate. When pyrophosphate is discriminated as being present, it is discriminated that the extension reaction of the primer proceeded. Further, it is discriminated that a DNA having a particular base sequence was present in the sample. On the other hand, when pyrophosphate is discriminated as not being present, it is discriminated that the extension reaction of the primer did not proceed. Further, it is discriminated that a DNA having a particular base sequence was not present in the sample. In summary, the presence/absence of a DNA having a particular base sequence can be discriminated. The method of qualitatively detecting pyrophosphate of this Embodiment is just the same as the method in Embodiment 1 described above, therefore, the explanation is now omitted.

As described hereinabove, the presence/absence of a nucleic acid having a particular base sequence in a sample can be discriminated by analyzing pyrophosphate that is produced in an amplification method of the nucleic acid having the particular base sequence in the sample, by way of analysis of the change of the H⁺ concentration using H⁺-pyrophosphatase Moreover, relative quantitative determination for a base sequence which shall be a standard of a particular base sequence can be also carried out through utilizing the method on this Embodiment, by executing en extension reaction of a primer using a primer that is complementary to a nucleic acid having a particular base sequence, and comparing the amount of pyrophosphate produced by the reaction with the amount of pyrophosphate produced when an extension reaction of a primer is executed using a primer having a sequence to be a standard.

It is possible to put the method of discriminating the presence/absence of a nucleic acid having a particular base sequence explained in this Embodiment into effect using the device for detecting pyrophosphate 50, the device for discriminating a base type 60, and the tip 53a, 53b or 90 which were explained in Embodiment 1 as described above.

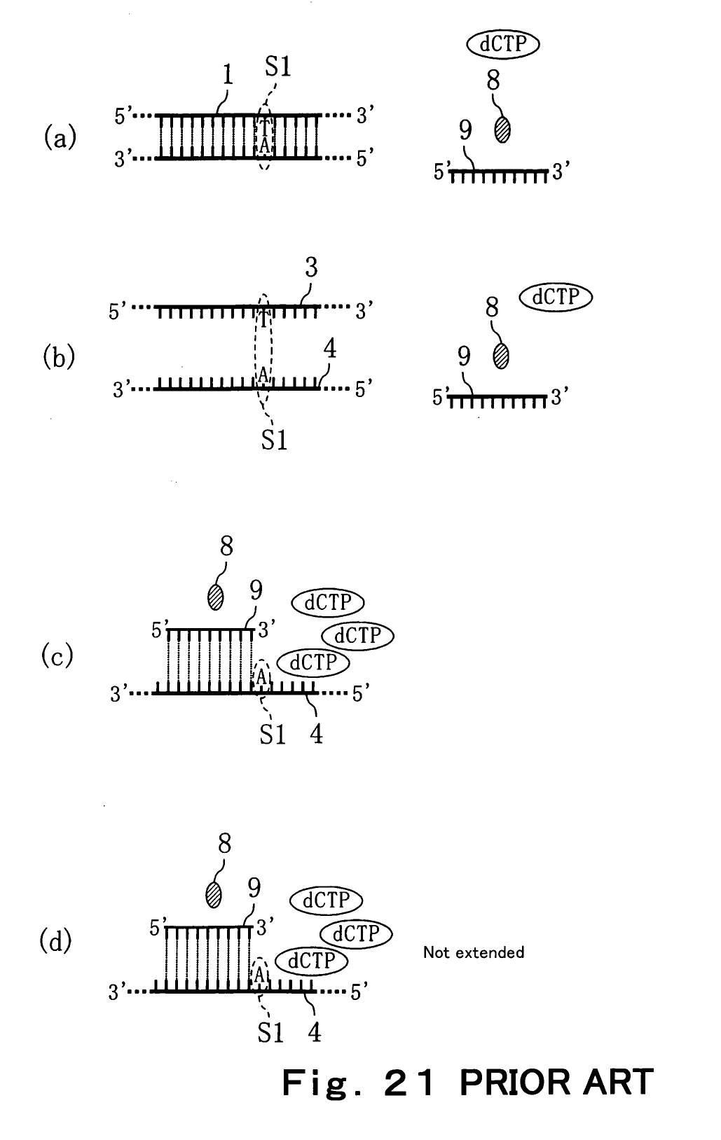

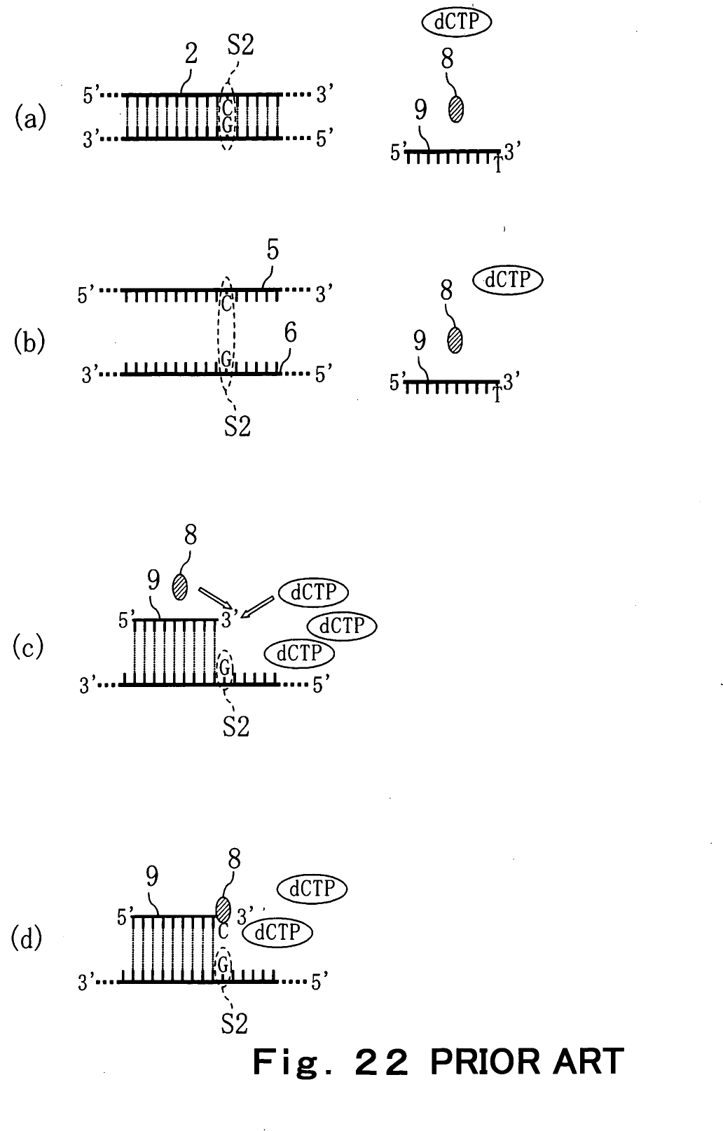

Although methods in which a primer extension reaction is utilized using 4 kinds of dNTPs are explained in the aforementioned Embodiments 1 and 2, as a matter of course, a primer extension reaction using one kind of dNTP (or ddNTP) can be also utilized, as explained with reference to FIGS. 21 and 22 in connection with the conventional technology. In addition, an amplification method of a nucleic acid having a particular base sequence, such as PCR or the like, in which two or more kinds of primers including a typing primer are used may be employed in combination. Moreover, also in respect of the typing primer, it is not limited to the primer having its 3′ end corresponding to the SNP site, and having a base sequence that is completely complementary to the base sequence adjacent to the SNP site, but any primer which can discriminate the base type on the basis of the extent of progress of the primer extension reaction is acceptable. For example, known primers such as: a primer having its 3′ end corresponding to a SNP site and having a base sequence that is completely complementary to the base sequence adjacent to the SNP site except for 1 base; a primer of which site adjacent to its 3′ end corresponds to a SNP site; and the like can be also used. In other words, amplification of a nucleic acid having a base sequence including a SNP which is an object of analysis may be analyzed using H⁺- pyrophosphatase to execute the discrimination of the base type of a SNP site.

As a matter of course, according to the method of the Embodiment 1 described above, it is possible to discriminate not alone the base type of a SNP site, but a particular base sequence can be also discriminated.

Additionally, in the Embodiments 1 and 2 described above, determination of the base type in a base sequence of a DNA and detection of DNA are explained, however, they are not limited to DNA, of course, but determination of the base type in a base sequence of an RNA and detection of RNA can be similarly performed. Furthermore, the sample which can be used includes any one irrespective of a single stranded DNA or a double stranded DNA.

(Detection Experiment of pyrophosphate 1)

This Example was conducted according to the method of Shizuo Yoshida et al., (Masayoshi Maeshima and Shizuo Yoshida, 1989, J. Biol. Chem., 264(33), pp. 20068-20073) as demonstrated below.

First, membrane vesicles comprising a tonoplast membrane derived from Phaseolus aureus was dissolved in a solution containing Tris/Mes buffer (concentration of 5 mM, pH 7.0), sorbitol (concentration of 0.25 M) and DTT (concentration of 2 mM) to give a suspension of membrane vesicles comprising a tonoplast membrane.

Next, this suspension was mixed in a reaction liquid containing MgSO₄ (concentration of 1 mM), KCl (concentration of 50 mM), sorbitol (concentration of 0.25 M), acridine orange (pH sensitive pigment, concentration of 3 μM), Hepes/Bristris propane (concentration of 25 mM, pH 7.2) to prepare a H⁺-pyrophosphatase liquid.

Next, this H⁺-pyrophosphatase liquid was evenly dispensed into 4 tubes, and thereto was added a sodium pyrophosphate solution such that each final concentration of sodium pyrophosphate became 10 μM, 20 μM, 40 μM, 60 μM, 80 μM and 100 μM, respectively, to initiate the hydrolysis reaction of pyrophosphate by H⁺-pyrophosphatase.

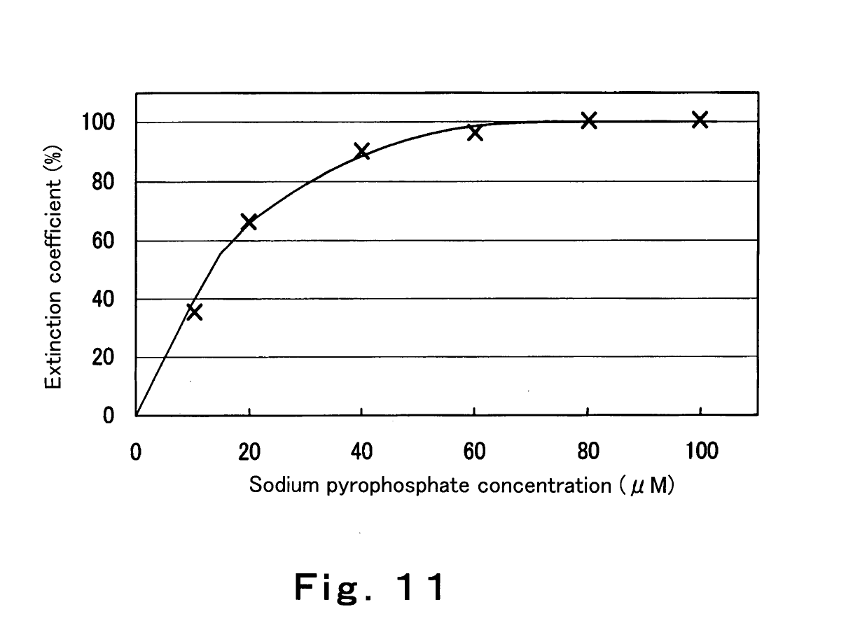

In this Example, an excitation light of 493 nm was irradiated on each reaction liquid, and the change of fluorescence intensity at 540 nm before and after adding the sodium pyrophosphate solution was analyzed. The results are shown in FIG. 11.

FIG. 11 is a graph showing the relationship between the concentration of sodium pyrophosphate and the change of fluorescence intensity at 540 nm. In this Figure, the change of fluorescence intensity at 540 nm is represented by extinction coefficient per unit second in the reaction liquid that corresponds to each sodium pyrophosphate concentration. Further, extinction coefficient per unit second in the reaction liquid that corresponds to each sodium pyrophosphate concentration is converted, on the basis of the extinction coefficient per unit second in the reaction liquid having the final concentration of sodium pyrophosphate of 100 μM assumed as 100%.

As is shown in FIG. 11, a result was obtained indicating that the extinction coefficient of acridine orange per 1 second is altered with a relationship with approximately hyperbolic function depending on the concentration of sodium pyrophosphate. Accordingly, it is revealed that pyrophosphate can be quantitatively detected by measuring the extinction coefficient of acridine orange per 1 second.

(Detection Experiment of pyrophosphate 2)

This Example was conducted according to the method of Masasuke Yoshida et al., (MasaH.Sato, Masahiko Kasahara, Noriyuki Ishii, Haruo Homareda, Hideo Matsui and Masasuke Yoshida, 1994, J. Biol. Chem., 269(9), pp. 6725-6728) as demonstrated below.

First, purification of tonoplast membrane H⁺-pyrophosphatase from seeds of squash was conducted.

Subsequently, a proteoliposome liquid of tonoplast membrane H⁺-pyrophosphatase was prepared by adding tonoplast membrane H⁺-pyrophosphatase obtained after purification into a lipid mixture that was prepared from phosphatidylcholine of soybean and cholesterol. After mixing this proteoliposome liquid in a reaction liquid containing sorbitol (concentration of 0.25 M), Tricine-Na (concentration of 10 mM, pH 7.5), EGTA (concentration of 0.1 mM), KCl (concentration of 50 mM) and Oxonol V (membrane potential sensitive pigment, concentration of 0.2 μM), the mixture was evenly dispensed into 5 tubes.

Subsequently, to the 5 tubes was added a sodium pyrophosphate solution such that final concentration of sodium pyrophosphate became 10 μM, 20 μM, 40 μM, 60 μM, 80 μM and 100 μM, respectively, to initiate the hydrolysis reaction of pyrophosphate by H⁺-pyrophosphatase.

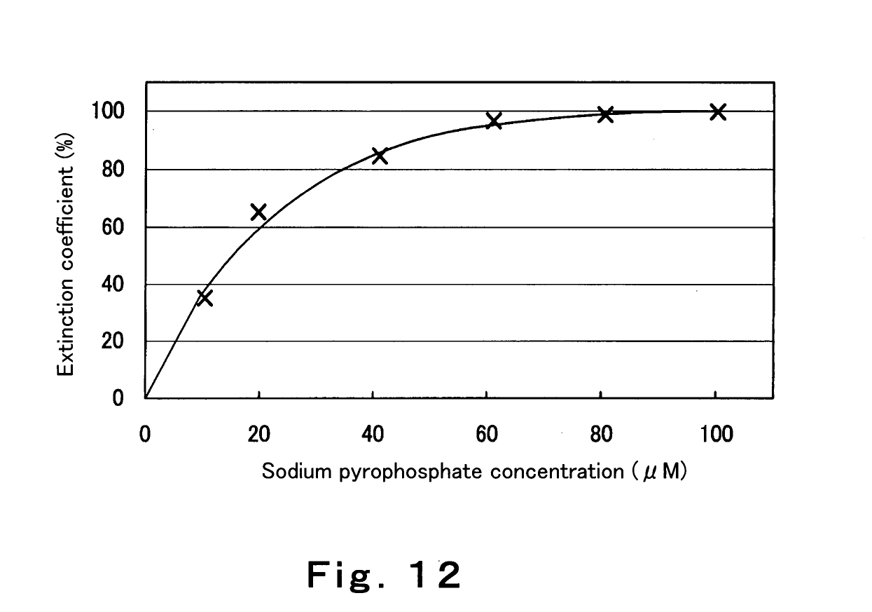

In this Example, an excitation light of 610 nm was irradiated on each reaction liquid, and alteration of membrane potential of proteoliposome included in each reaction liquid before and after adding the sodium pyrophosphate solution was analyzed by measuring the change of fluorescence intensity at 639 nm before and after adding the sodium pyrophosphate solution. The results are shown in FIG. 12.

FIG. 12 is a graph showing the relationship between the concentration of sodium pyrophosphate and the change of fluorescence intensity at 639 nm. In this Figure, the change of fluorescence intensity at 639 nm is represented by extinction coefficient per unit second in the reaction liquid that corresponds to each sodium pyrophosphate concentration. Further, extinction coefficient per unit second in the reaction liquid that corresponds to each sodium pyrophosphate concentration is converted, on the basis of the extinction coefficient per unit second in the reaction liquid having the final concentration of sodium pyrophosphate of 100 μM assumed as 100%.

As is shown in FIG. 12, a result was obtained indicating that the extinction coefficient of Oxonol V per 1 second is altered with a relationship with approximately hyperbolic function depending on the concentration of sodium pyrophosphate. Accordingly, it is revealed that pyrophosphate can be quantitatively detected by measuring the extinction coefficient of Oxonol V per 1 second.

(Detection Experiment of pyrophosphate 3)

This Example was conducted according to the method disclosed in JP-A No. 6-90736.

First, similarly to Example 2 described above, a lipid bilayer including tonoplast membrane H⁺-pyrophosphatase was fixed on a commercially available ISFET-pH sensor using tonoplast membrane H⁺-pyrophosphatase derived from seeds of squash. It should be noted however that outside of the lipid bilayer was filled with a reaction solution containing MgSO₄ (concentration of 1 mM), KCl (concentration of 50 mM), sorbitol (concentration of 0.25 M), Hepes/Bristris propane (concentration of 25 mM, pH 7.2).

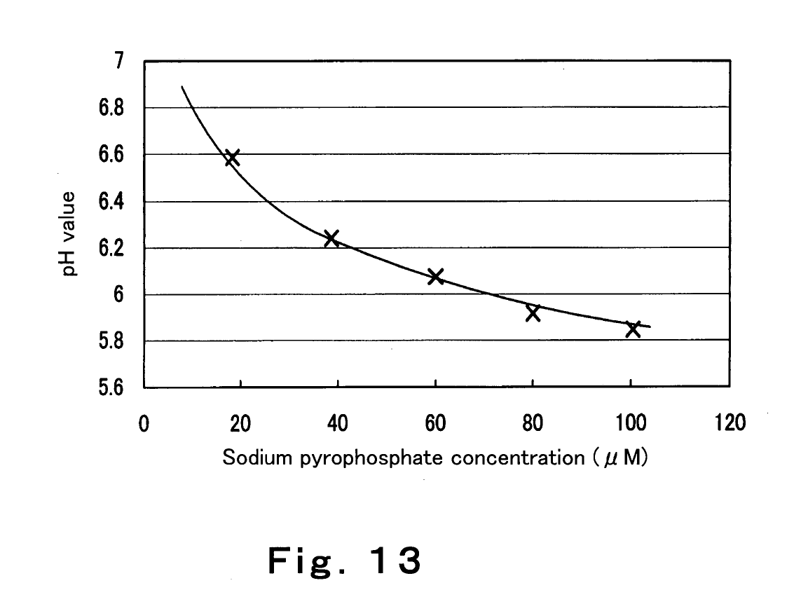

Next, using the aforementioned ISFET-pH sensor with the fixed lipid bilayer including tonoplast membrane H⁺-pyrophosphatase, each pH value was measured in instances where a sodium pyrophosphate solution was added such that final concentration of sodium pyrophosphate in the aforementioned reaction solution became 20 μM, 40 μM, 60 μM, 80 μM and 100 μM, respectively. The results are shown in FIG. 13.

As is shown in FIG. 13, a result was obtained indicating that the pH value is decreased depending on the concentration of sodium pyrophosphate. Accordingly, it is revealed that pyrophosphate can be quantitatively detected by measuring the pH value.

### EXAMPLE 1

In this Example, detection of λDNA (with regard to entire base sequence of λDNA, see, Accession No. V00636, J02459, M17233 and X00906 of GenBank database) in a sample was conducted.

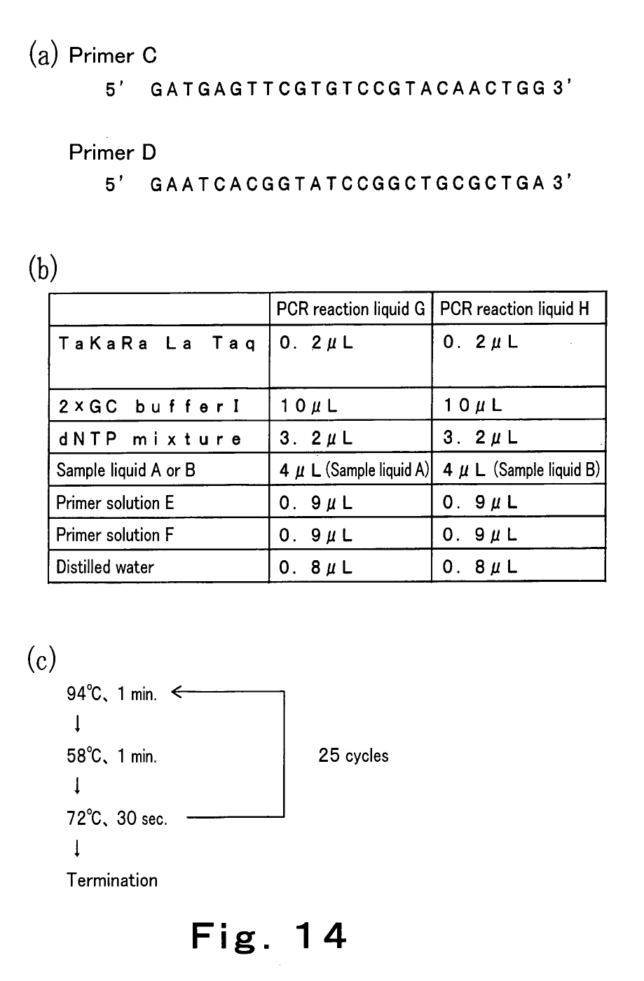

First, a sample liquid A containing λDNA (manufactured by Takara Shuzo Co., Ltd.) at the concentration of 10 ng/μL dissolved in distilled water, and a sample liquid B consisting of distilled water alone were provided. Also, as shown in FIG. 14 (a), primer solutions E and F containing two kinds of primers C and D, which can completely hybridize to a particular base sequence of λDNA, dissolved in distilled water (20 μM each), respectively, were provided.

To the aforementioned sample liquids A and B were respectively added TaKaRa La Taq (5 U/μL, manufactured by Takara Shuzo Co., Ltd.), 2×GC buffer I that is a buffer for exclusive use of TaKaRa La Taq (manufactured by Takara ShuzoCo., Ltd.), a dNTP mixture (each concentration of 2.5 mM, manufactured by Takara Shuzo Co., Ltd.), and primer solutions E and F to prepare the PCR reaction liquids G and H having the composition presented in FIG. 14 (b).

Next, for each of the PCR reaction liquids G and H, a PCR reaction was conducted under the reaction temperature conditions presented in FIG. 14 (c).

After terminating the PCR reaction, each of the PCR reaction liquids G and H was mixed with the H⁺-pyrophosphatase liquid described in the above Example 1 to subject to a reaction.

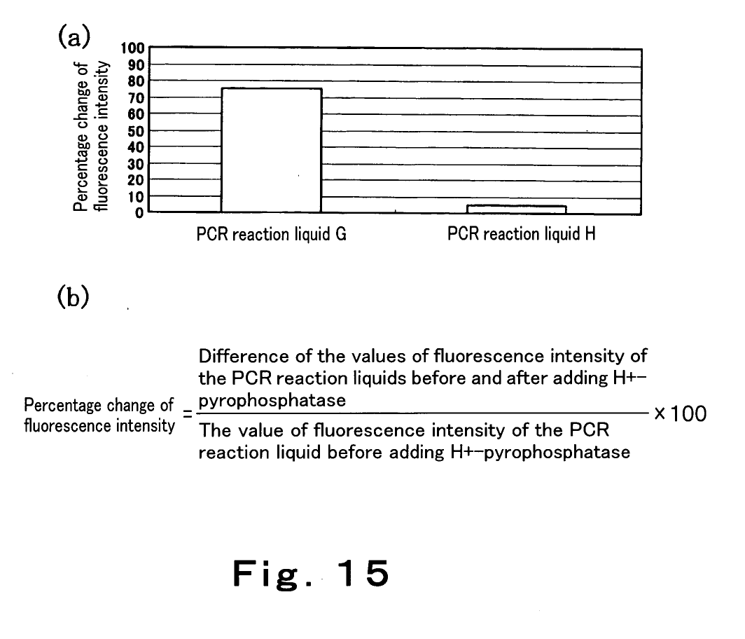

In this Example, the change of fluorescence intensity of acridine orange before and after mixing the H⁺-pyrophosphatase liquid was analyzed for each of the PCR reaction liquids G and H. For the analysis of fluorescence intensity of acridine orange, an excitation light of 493 nm was irradiated, and the analysis was performed with respect to fluorescence intensity at 540 nm. The results are shown in FIG. 15 (a).

FIG. 15 (a) illustrates the percentage change of fluorescence intensity before and after mixing the H⁺-pyrophosphatase liquid to each of the PCR reaction liquids G and H, respectively. The percentage change of fluorescence intensity is represented by the formula shown in FIG. 15 (b).

As is shown in FIG. 15 (a), the percentage change of fluorescence intensity of the PCR reaction liquid G is evidently greater than that of the PCR reaction liquid H. In other words, it is proven that pyrophosphate was produced in the PCR reaction liquid G, indicating that the primer extension reaction proceeded. On the grounds of such a result, it is discriminated that the target nucleic acid was present in the PCR reaction liquid G. Accordingly, it is revealed that a target nucleic acid can be detected by measuring the fluorescence intensity of acridine.

### EXAMPLE 2

In this Example, mutant λDNA involving an artificial substitution of a certain base in the base sequence of λDNA into other base was generated, and studied as to whether or not discrimination can be executed between normal λDNA and the mutant λDNA.

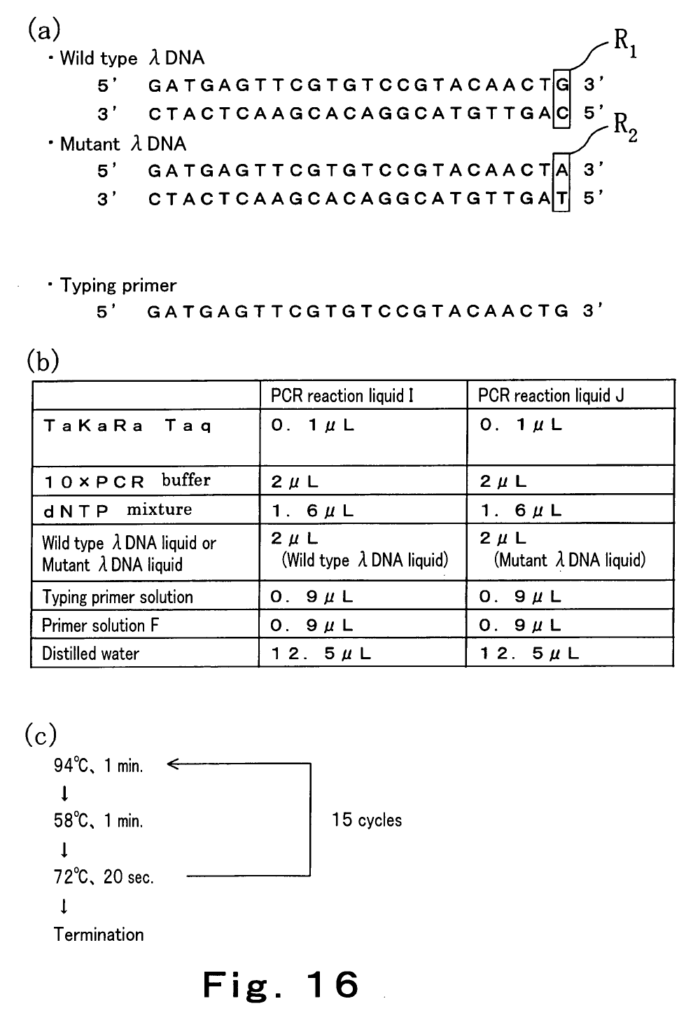

First, the mutant λDNA was generated using λDNA (manufactured by Takara Shuzo Co., Ltd.). A GC base pair (in the Figure, region R₁) in λDNA illustrated in FIG. 16 (hereinafter, normal λDNA is described as wild type λDNA) which is present in the double stranded DNA sequence was artificially substituted with an AT base pair (in the Figure, region R₂) by a well known method to persons skilled in the art to give the mutant λDNA.

Next, the wild type λDNA and mutant λDNA were dissolved in distilled water to give the final concentration of 10 ng/μL, respectively, to prepare a wild type λDNA liquid and a mutant λDNA liquid, respectively.

Next, in order to discriminate the difference of the bases described above, a typing primer illustrated in FIG. 16 (a) was provided. Subsequently, a typing primer solution was prepared by dissolving the typing primer in distilled water to give the final concentration of 20 μM.

The typing primer illustrated in FIG. 16 (a) completely hybridizes to the single stranded DNA described in the lower panel of wild type λDNA. However, the base G at 3′ end of this typing primer can not hybridize to the single stranded DNA described in the lower panel of mutant λDNA. Therefore, when the primer extension reaction is executed using this typing primer, the reaction satisfactorily proceeds in the instance of wild type λDNA, however, the reaction does not proceed well in the instance of mutant λDNA.

Also, the primer solution F used in the aforementioned Example 4 was provided.

Next, for each of the wild type λDNA liquid and the mutant λDNA liquid, the PCR reaction liquids I and J having the composition presented in FIG. 16 (b) were prepared using TaKaRa Taq (5 U/μL, manufactured by Takara Shuzo Co., Ltd.), 10×PCR buffer that is for exclusive use of TaKaRa Taq (manufactured by Takara Shuzo Co., Ltd.), a dNTP mixture (each concentration of 2.5 mM, manufactured by Takara Shuzo Co., Ltd.), and the typing primer solution and the primer solution F.

Next, in the PCR reaction liquids I and J, a PCR reaction was conducted, respectively, under the reaction temperature conditions presented in FIG. 16 (c).

After terminating the PCR reaction, the PCR reaction liquids I and J were respectively mixed with the H⁺-pyrophosphatase liposome liquid to subject to a reaction. The H⁺-pyrophosphatase liposome liquid was prepared according to the method of Masasuke Yoshida et al, (MasaH.Sato, Masahiko Kasahara, Noriyuki Ishii, Haruo Homareda, Hideo Matsui and Masasuke Yoshida., 1994, J. Biol. Chem., 269(9), pp. 6725-6728).

Specifically, purification of tonoplast membrane H⁺-pyrophosphatase from seeds of squash was first conducted. Subsequently, a proteoliposome liquid of tonoplast membrane H⁺-pyrophosphatase was prepared by adding tonoplast membrane H⁺-pyrophosphatase obtained after purification into a lipid mixture that was prepared from phosphatidylcholine of soybean and cholesterol. This proteoliposome liquid was mixed in a reaction liquid containing sorbitol (concentration of 0.25 M), Tricine-Na (concentration of 10 mM, pH 7.5), EGTA (concentration of 0.1 mM), KCl (concentration of 50 mM) and Oxonol V (membrane potential sensitive pigment, concentration of 0.2 μM), to give a H⁺-pyrophosphatase liposome liquid.

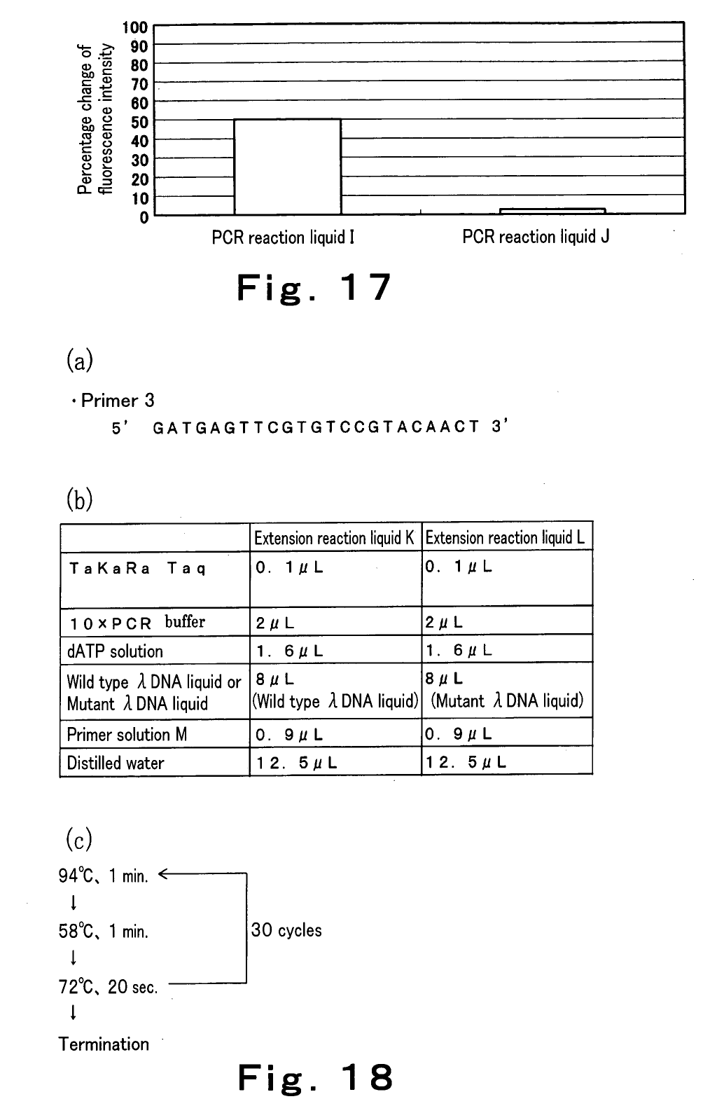

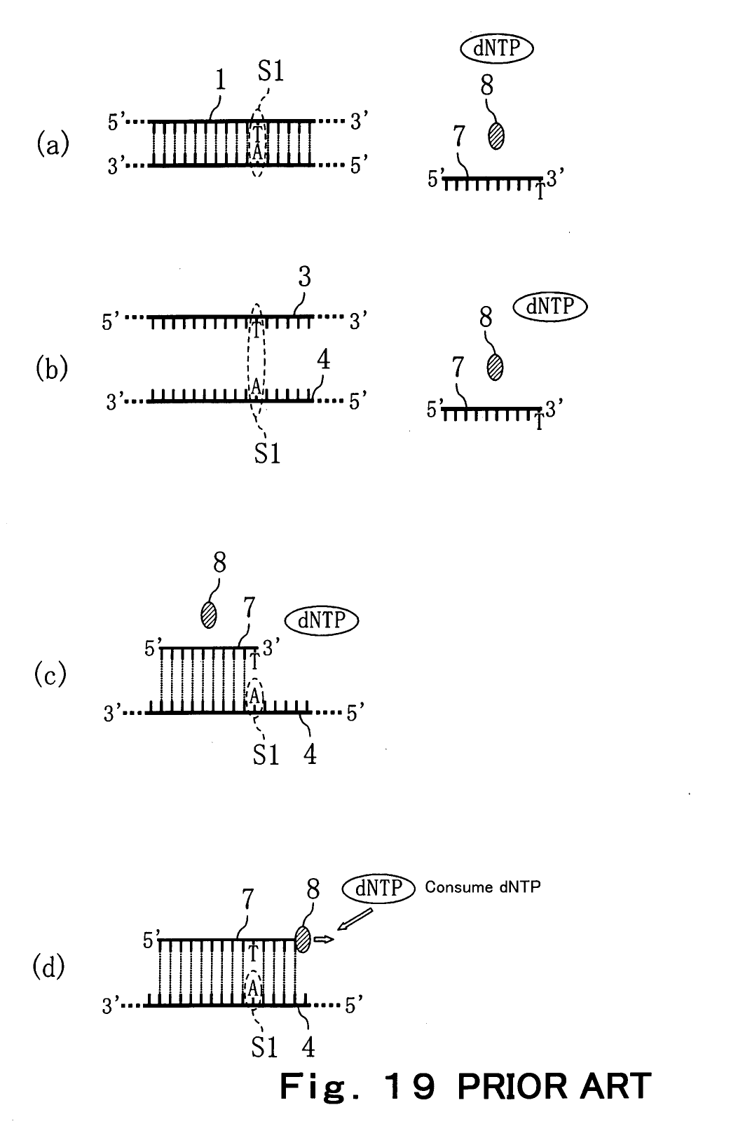

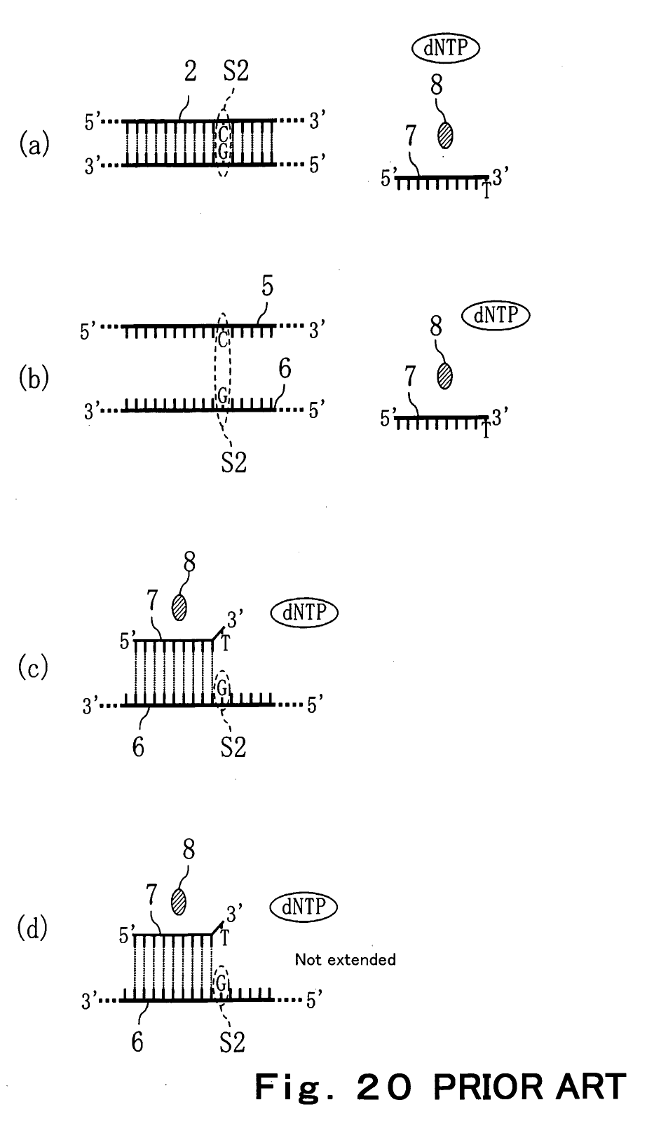

In this Example, an excitation light of 610 nm was irradiated on each PCR reaction liquid, and alteration of membrane potential of proteoliposome included in each reaction liquid was analyzed by measuring the change of fluorescence intensity at 639 nm of Oxonol V before and after adding the sodium pyrophosphate solution. The results are shown in FIG. 17.

FIG. 17 illustrates the percentage change of fluorescence intensity before and after mixing of the PCR reaction liquids I and J, respectively. As is shown in FIG. 17, the percentage change of fluorescence intensity of the PCR reaction liquid I is evidently greater than that of the mutant PCR reaction liquid J. The grounds therefore are believed that the PCR reaction did not proceed well in the PCR reaction liquid J, however, the reaction satisfactorily proceeded in the PCR reaction liquid I, and consequently, thus produced pyrophosphate reacted with H⁺-pyrophosphatase that was present in liposome, leading to the transport of H⁺ into liposome.

Therefore, according to this Example, it is proven that a difference of a single base pair in a particular base sequence of DNA can be discriminated. In other words, it is suggested that the method of this Example is extremely effective for discriminating the base type of a SNP site, and for discriminating a particular base type such as mutation of a single base pair caused by a discontinuous variation.

### EXAMPLE 3

In this Example, unlike the Example 5 described above, possible discrimination of the difference of a single base pair between wild type λDNA and mutant λDNA was studied with a method of combination of a 1 base extension reaction and a reaction of H⁺-pyrophosphatase.

First, a wild type λDNA (5 mM) liquid and a mutant XDNA (5 mM) liquid were prepared by dissolving the same wild type λDNA and mutant λDNA as those used in the aforementioned Example 5 in distilled water to give the final concentration of 5 mM.

Next, the primer illustrated in FIG. 18 (a) was provided. This primer can completely hybridize to the single stranded DNA shown in the lower panel of wild type λDNA which is illustrated in FIG. 16 (a) in Example 5, at the sequence other than the base C at its 5′ end. In other words, similarly to the single stranded DNA sequence shown in the lower panel of the mutant λDNA sequence demonstrated in Example 5, it can completely hybridize to the sequence other than the base T at its 5′ end.

Next, a primer solution M was prepared in which this primer was dissolved in distilled water to give the final concentration of 0.2 mM.

Subsequently, for each of the wild type λDNA (5 mM) liquid and the mutant λDNA (5 mM) liquid, the extension reaction liquids K and L having the composition presented in FIG. 18 (b) were prepared using TaKaRa Taq (5 U/μL, manufactured by Takara Shuzo Co., Ltd.) and 10×PCR buffer that is for exclusive use of TaKaRa La Taq (manufactured by Takara Shuzo Co., Ltd.), and a 2.5 mM dATP solution and the primer solution M.

Subsequently, for each of the extension reaction liquids K and L, a single base extension reaction was conducted under the reaction temperature conditions presented in FIG. 18 (c).

After terminating the single base extension reaction, each extension reaction liquid was introduced to a modified ISFET electrode including H⁺-pyrophosphatase fixed thereto. The modified ISFET electrode was that used in the aforementioned Example 3.

Using this modified ISFET electrode, each pH value was measured in the instance of each extension reaction liquid was added. As a result, the pH of the extension reaction liquid K was 6.89, whilst the pH of the extension reaction liquid L was 6.02. Grounds for this result are believed that the extension reaction did not occur in the extension reaction liquid K containing wild type λDNA, whilst the single base extension reaction by dATP occurred in the extension reaction liquid L containing mutant λDNA, and consequently, thus produced pyrophosphate reacted with H⁺-pyrophosphatase on the modified ISFET electrode, leading to the transport of H⁺ to the modified ISFET electrode side.

According to this method, it is proven that a difference of a single base pair in a base sequence of a target nucleic acid can be discriminated. In other words, it is revealed that this method is an extremely effective method for discriminating the base type of a SNP site, and for discriminating a particular base sequence such as substitution of a single base pair caused by a discontinuous variation.

According to the present invention, convenient techniques for detecting an extension reaction of a primer, convenient techniques for discriminating the base type in a base sequence of a nucleic acid, convenient techniques for detecting pyrophosphate, and convenient techniques for detecting a nucleic acid having a particular base sequence can be provided.

From the description hereinabove, many modifications and other embodiments will be apparent to persons skilled in the art. Therefore, the above description should be construed as merely illustration exemplification, which are provided for the purpose of teaching the best embodiment for carrying out the present invention. Without departing from the spirit of the present invention, details of the structure and/or function thereof can be substantially altered.

1. A method of detecting an extension reaction in which an extension reaction of a primer is detected, said method comprises the following steps of:
the step (a) of preparing a sample solution containing a nucleic acid, a primer having a base sequence that includes a complementary binding region which complementarily binds to said nucleic acid, and a nucleotide;
the step (b) of allowing said sample solution to stand under a condition to cause said extension reaction, and producing pyrophosphate when said extension reaction is caused;
the step (c) of bringing said sample solution into contact with the front face of a H⁺ hardly permeable membrane having H⁺-pyrophosphatase, which penetrates from front to back of the membrane, of which active site that hydrolyzes pyrophosphate being exposed to the front face;
the step (d) of measuring the H⁺ concentration of at least either one of the solution at the front face side of said H⁺ hardly permeable membrane or the solution at the back face side of said H⁺ hardly permeable membrane, in a state where said H⁺-pyrophosphatase is immersed in the solution; and
the step (e) of detecting said extension reaction on the basis of the result of measurement in the step (d).

2. A method of discriminating a base type in which the base type in a base sequence of a nucleic acid is discriminated, said method comprises the following steps of:
the step (a) of preparing a sample solution containing a nucleic acid, a primer having a base sequence that includes a complementary binding region which complementarily binds to said nucleic acid, and a nucleotide;
the step (b) of allowing said sample solution to stand under a condition to cause an extension reaction of said primer, and producing pyrophosphate when said extension reaction is caused;
the step (c) of bringing said sample solution into contact with the front face of a H⁺ hardly permeable membrane having H⁺-pyrophosphatase, which penetrates from front to back of the membrane, of which active site that hydrolyzes pyrophosphate being exposed to the front face;
the step (d) of measuring the H⁺ concentration of at least either one of the solution at the front face side of said H⁺ hardly permeable membrane or the solution at the back face side of said H⁺ hardly permeable membrane, in a state where said H⁺-pyrophosphatase is immersed in the solution;
the step (e) of detecting said extension reaction on the basis of the result of measurement in the step (d); and
the step (f) of discriminating the base type in the base sequence of said nucleic acid on the basis of the result of detection in the step (e).

3. The method of discriminating a base type according to claim 2 wherein the difference between the H⁺ concentration of the solution at said front face side, and the H⁺ concentration of said sample solution post the step (b) and before the step (c) is measured, in the step (d).

4. The method of discriminating a base type according to claim 3 wherein said extension reaction is detected by comparing the result of measurement in the step (d) with a control value, in the step (e).

5. The method of discriminating a base type according to claim 4 wherein said discrimination of a base type is the discrimination of the base type of a SNP site, and
said control value is the result of measurement obtained in the step (d) through carrying out the steps (a), (b), (c) and (d) using a nucleic acid having said SNP site without mutation, as said nucleic acid.

6. The method of discriminating a base type according to claim 2 wherein the H⁺ concentration of the solution at said back face side is detected in the step (d), and
said extension reaction is detected by comparing the result of measurement in the step (d) with a control value, in the step (e).

7. The method of discriminating a base type according to claim 6 wherein said discrimination of a base type is the discrimination of the base type of a SNP site,
one kind of a nucleotide is used as said nucleotide in the step (a), and
said control value is the result of measurement obtained in the step (d) through carrying out the steps (a), (b), (c) and (d) using a nucleic acid having said SNP site with a different base type, as said nucleic acid.

8. The method of discriminating a base type according to claim 2 wherein said H⁺ concentration is optically measured in the step (d).

9. The method of discriminating a base type according to claim 8 wherein a pH sensitive pigment or a membrane potential sensitive pigment is added to at least either one of the solution at said front face side and the solution at the back face side, in the step (d).

10. The method of discriminating a base type according to claim 9 wherein acridine orange or Oxonol is added to at least either one of the solution at said front face side and the solution at the back face side, in the step (d).

11. The method of discriminating a base type according to claim 2 wherein said H⁺ concentration is electrically measured in the step (d).

12. The method of discriminating a base type according to claim 2 wherein said extension reaction is an extension reaction according to a PCR method.

13. A device for discriminating a base type in which the base type in a base sequence of a nucleic acid is discriminated which comprises:
a reaction section in which thermoregulation required for an extension reaction of a primer is carried out, and
a pyrophosphate detection section in which pyrophosphate that is produced by said primer extension reaction is detected, wherein said reaction section is provided with a reserving region for reaction where a solution is reserved,
said pyrophosphate detection section is provided with a reserving region for detection where a solution is reserved, a H⁺ hardly permeable membrane that separates said reserving region for detection to a first region and a second region, and a measurement means for measuring the H⁺ concentration of the solution reserved in at least either one of the first region and second region, and
wherein said H⁺ hardly permeable membrane has H⁺-pyrophosphatase, which penetrates from front to back of the membrane, of which active site that hydrolyzes pyrophosphate being exposed to the front face, and
in said pyrophosphate detection section, the reaction solution which is delivered from said reaction section is reserved in the first region.

14. The device for discriminating a base type according to claim 13 wherein said measurement means optically measures the H⁺ concentration.

15. The device for discriminating a base type according to claim 13 wherein said measurement means electrically measures the H⁺ concentration.

16. The device for discriminating a base type according to claim 13 further comprising an analysis means for controlling said reaction section and said pyrophosphate detection section, and for analyzing the result of measurement from said measurement means.

17. The device for discriminating a base type according to claim 13 further comprising a slot to which a tip can be inserted that is provided with said reserving region for reaction and said reserving region for detection.

18. A device for detecting pyrophosphate which comprises a vessel, a H⁺ hardly permeable membrane that separates inside of said vessel into a first region and a second region, an electrode that is provided such that it is brought into contact with a solution reserved in the first region, and a H⁺ sensitive electrode that is provided such that it is brought into contact with a solution reserved in the second region,
wherein said H⁺ hardly permeable membrane has H⁺-pyrophosphatase, which penetrates from front to back of the membrane, of which active site that hydrolyzes pyrophosphate being exposed to the front face.

19. A method of detecting a nucleic acid having a particular base sequence, said method comprises the following steps of:
the step (a) of preparing a sample solution containing a sample, a primer having a base sequence that includes a complementary binding region which complementarily binds to said nucleic acid, and a nucleotide;
the step (b) of allowing said sample solution to stand under a condition to cause an extension reaction of said primer, and producing pyrophosphate when said extension reaction is caused;
the step (c) of bringing said sample solution into contact with the front face of a H⁺ hardly permeable membrane having H⁺-pyrophosphatase, which penetrates from front to back of the membrane, of which active site that hydrolyzes pyrophosphate being exposed to the front face;
the step (d) of measuring the H⁺ concentration of at least either one of the solution at the front face side of said H⁺ hardly permeable membrane or the solution at the back face side of said H⁺ hardly permeable membrane, in a state where said H⁺-pyrophosphatase is immersed in the solution;
the step (e) of detecting said extension reaction on the basis of the result of measurement in the step (d); and
the step (f) of detecting the nucleic acid on the basis of the result of detection in the step (e).

20. The method of detecting a nucleic acid according to claim 19 wherein the difference between the H⁺ concentration of the solution at said front face side and the H⁺ concentration of said sample solution post the step (b) and before the step (c) is measured in the step (d).

21. The method of detecting a nucleic acid according to claim 20 wherein said extension reaction is detected by comparing the result of measurement in the step (d) with a control value, in the step (e).

22. The method of detecting a nucleic acid according to claim 21 wherein said control value is the result of measurement obtained in the step (d) through carrying out the steps (a), (b), (c) and (d) using said sample without including a nucleic acid.

23. The method of detecting a nucleic acid according to claim 19 wherein said H⁺ concentration is optically measured in the step (d).

24. The method of detecting a nucleic acid according to claim 23 wherein a pH sensitive pigment or a membrane potential sensitive pigment is added to at least either one of the solution at said front face side and the solution at said back face side, in the step (d).

25. The method of detecting a nucleic acid according to claim 24 wherein acridine orange or Oxonol V is added to at least either one of the solution at said front face side and the solution at said back face side, in the step (d).

26. The method of detecting a nucleic acid according to claim 19 wherein said H⁺ concentration is electrically measured in the step (d).

27. The method of detecting a nucleic acid according to claim 19 wherein said extension reaction is an extension reaction according to a PCR method.

28. A tip for introducing a sample solution which comprises a reaction chamber for carrying out an extension reaction of a primer, a pyrophosphate detection chamber for detecting pyrophosphate, and a flow pass that connects said reaction chamber and said pyrophosphate detection chamber.

29. The tip for introducing a sample solution according to claim 28 wherein said flow pass can be opened and closed.

30. The tip for introducing a sample solution according to claim 28 wherein said pyrophosphate detection chamber has a first region and a second region which are separated by a H⁺ hardly permeable membrane;
said H⁺ hardly permeable membrane has H⁺-pyrophosphatase, which penetrates from front to back of the membrane, of which active site that hydrolyzes pyrophosphate being exposed to the front face; and
in said pyrophosphate detection chamber, the reaction solution that is delivered from said reaction chamber via said flow pass is reserved in the first region.


SEQUENCE LISTING (6 sequences)

SEQ ID NO: 1 - DNA, 25 bp, Artificial Sequence
  Chemically synthesized
gatgagttcgtgtccgtacaactgg

SEQ ID NO: 2 - DNA, 25 bp, Artificial Sequence
  Chemically synthesized
gaatcacggtatccggctgcgctga

SEQ ID NO: 3 - DNA, 24 bp, Artificial Sequence
  Chemically synthesized
gatgagttcgtgtccgtacaactg

SEQ ID NO: 4 - DNA, 24 bp, Artificial Sequence
  Chemically synthesized
gatgagttcgtgtccgtacaacta

SEQ ID NO: 5 - DNA, 24 bp, Artificial Sequence
  Chemcially synthesized
gatgagttcgtgtccgtacaactg

SEQ ID NO: 6 - DNA, 23 bp, Artificial Sequence
  Chemically synthesized
gatgagttcgtgtccgtacaact